(19) United States
(12) Patent Application Publication
(10) Pub. No.: US 2014/0329903 A1
(43) Pub. Date: Nov. 6, 2014

(54) TREATMENT OF NEURODEGENERATIVE DISEASES

(71) Applicants: Ethan S. Burstein, San Diego, CA (US); Roger Olsson, Bunkeflostrand (SE); Krista M. McFarland, San Marcos, CA (US)

(72) Inventors: Ethan S. Burstein, San Diego, CA (US); Roger Olsson, Bunkeflostrand (SE); Krista M. McFarland, San Marcos, CA (US)

(73) Assignee: ACADIA PHARMACEUTICALS INC., San Diego, CA (US)

(21) Appl. No.: 14/237,151

(22) PCT Filed: Aug. 7, 2012
(86) PCT No.: PCT/EP2012/065392

§ 371 Date: May 13, 2014

Related U.S. Application Data
(60) Provisional application No. 61/522,548, filed on Aug. 11, 2011.

(30) Foreign Application Priority Data
Aug. 11, 2011 (EP) 11177210.9

(51) Int. Cl. A61K 31/192

(52) U.S. Cl. CPC A61K 31/192
USPC 514/569

(57) ABSTRACT

The present disclosure relates to compounds to be used in a low dose in treatment of neurodegenerative diseases or disorders. It also relates to methods for treatment of a neurodegenerative diseases or disorders.

### FIELD

Provided herein are compounds that affect Nurr1 receptors and methods of using such compounds for modulating neurodegenerative conditions.

### BACKGROUND

Nuclear receptor related 1 protein (NURR1) also known as NR4A2 (nuclear receptor subfamily 4, group A, member 2), henceforth Nurr1 is a nuclear hormone receptor (NucHR) strongly implicated in the growth, maintenance, and survival of dopaminergic neurons, that represents a very promising therapeutic target for Parkinson's disease (PD). The essential role of Nurr1 in dopaminergic cell development was dramatically demonstrated in mouse gene knockout experiments in which homozygous mice lacking Nurr1 failed to generate midbrain dopaminergic neurons (Zetterstrom et al., 1997). Nurr1 was shown to be directly involved in the regulation of genes coding for aromatic amino acid decarboxylase, tyrosine hydroxylase (TH), and the dopamine transporter (DAT) (Hermanson et al., 2003). In addition, Nurr1 limits inflammatory responses in the central nervous system (CNS) and specifically protects dopaminergic neurons from neurotoxicity (Saijo et al., 2009). These observations suggest that Nurr1 play a pathophysiological role in aspects of neurodegenerative diseases ranging from inflammatory responses to dopaminergic nerve function and survival.

For example Nurr1 agonists have great potential as Parkinson's drugs as they enhance TH and DAT expression in primary mesencephalic cultures and exert a beneficial effect on dopaminergic neurons in animal models of PD (Ordentlich et al., 2003; Jankovic et al., 2005; Dubois et al., 2006). However, the molecular basis for the actions of existing ligands is not well defined. Nurr1 may mediate its beneficial effects alone, or more likely in concert with other nuclear hormone receptor partners (Sacchetti et al., 2006; Carpentier et al., 2008). To date, there are a few examples of such ligands available for experimental testing (Shi, 2007).

Nurr1 can form dimers and is known to associate with other NucHRs including peroxisome proliferator-activated receptor gamma (PPARγ), glucocorticoid receptor (GR), farnesoid X receptor (FXR), and retinoid X receptor (RXR) (Sacchetti et al., 2006; Carpentier et al., 2008). It is currently unknown which Nurr1 interaction is therapeutically important in the treatment of PD. However, it is agreed that Nurr1 involvement in dopaminergic neuronal activation and cell survival is important (Shi, 2007). Several of the most potent Nurr1 binding compounds enhance TH and DAT expression in primary mesencephalic cultures and exert a beneficial effect on dopaminergic neurons in animal models of PD (Jankovic et al., 2005).

Accordingly, there is a need for compounds, such as Nurr1 agonists, or compounds that induce activation of Nurr1 indirectly through Nurr1 binding partners that are neuroprotective via activity at the Nurr1 receptor in the central nervous system, both as pharmacological tools and as therapeutic agents.

WO 2011/057022, WO 2009/146218, WO 2009/146216 and WO 2008/064133 all mention the compound bexarotene.

### SUMMARY

Some embodiments relate to a compound of formula (I)

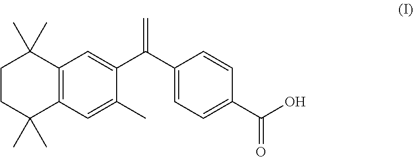 [SMILES: C=C(c1ccc(C(=O)O)cc1)c1cc2c(cc1C)C(C)(C)CCC2(C)C]

or a pharmaceutically acceptable salt, solvate, polymorph or hydrate thereof, for use in the treatment of a neurodegenerative disease or disorder wherein said compound is to be administered in a low dose.

Some embodiments relate to the use of a compound of formula (I)

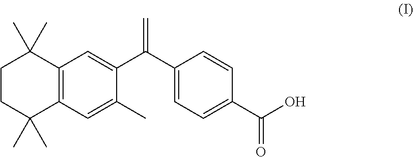 [SMILES: C=C(c1ccc(C(=O)O)cc1)c1cc2c(cc1C)C(C)(C)CCC2(C)C]

or a pharmaceutically acceptable salt, solvate, polymorph or hydrate thereof, for the manufacture of a pharmaceutical composition for use in the treatment of a neurodegenerative disease or disorder wherein said compound is to be administered in a low dose.

Some embodiments relate to a method for the treatment of a neurodegenerative disease or disorder, comprising the administration to a patient having a neurodegenerative disease or disorder an effective amount of the compound of formula (I)

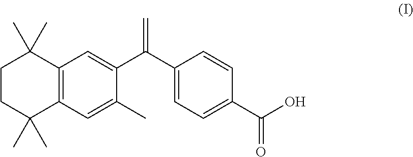 [SMILES: C=C(c1ccc(C(=O)O)cc1)c1cc2c(cc1C)C(C)(C)CCC2(C)C]

or a pharmaceutically acceptable salt, solvate, polymorph or hydrate thereof, wherein the compound is administered to the patient at a low dose.

Some embodiments relate to a method for the regeneration of the function of neurons in a patient having a neurodegenerative disease or disorder, comprising the administration to the patient having a neurodegenerative disease or disorder an effective amount of the compound of formula (I)

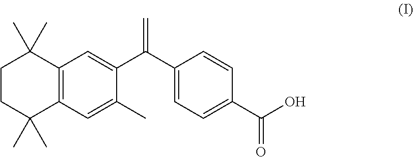 [SMILES: C=C(c1ccc(C(=O)O)cc1)c1cc2c(cc1C)C(C)(C)CCC2(C)C]

or a pharmaceutically acceptable salt, solvate, polymorph or hydrate thereof.

Some embodiments relate to a method for the protection of neurons in a patient having a neurodegenerative disease or disorder, comprising the administration to the patient having a neurodegenerative disease or disorder an effective amount of the compound of formula (I)

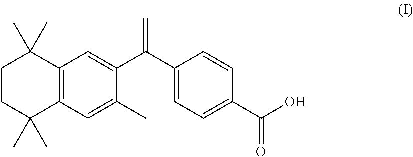 [SMILES: C=C(c1ccc(C(=O)O)cc1)c1cc2c(cc1C)C(C)(C)CCC2(C)C]

or a pharmaceutically acceptable salt, solvate, polymorph or hydrate thereof wherein the compound is administered to the patient at a dose low dose.

Some embodiments provide a pharmaceutical composition, comprising an effective amount of bexarotene (the compound of formula (I)) or a pharmaceutically acceptable salt, solvate, polymorph or hydrate thereof.

### DETAILED DESCRIPTION OF EMBODIMENTS

### Definitions

Unless defined otherwise, all technical and scientific terms used herein have the same meaning as is commonly understood by one of ordinary skill in the art. All patents, applications, published applications and other publications referenced herein are incorporated by reference in their entirety. In the event that there is a plurality of definitions for a term herein, those in this section prevail unless stated otherwise

The term “neurodegenerative disease or disorder” as used herein refers to a disease or disorder selected from the group consisting of Parkinson's disease, Alzheimer's disease, Huntington's disease, frontotemporal lobar degeneration associated with protein TDP-43 (FTLD-TDP, Dementia with Lewy bodies (DLB), vascular dementia, Amyotrophic lateral sclerosis (ALS), and other neurodegenerative related dementias due to changes in the brain caused by ageing, disease or trauma; or spinal cord injury. Such neurodegenerative diseases or disorders may be associated with a Nurr1 receptor.

The term “neuroprotection” as used herein refers to the prevention of further loss of neuronal cells, or loss of neuronal function as a result of exposure to a neurotoxin or resulting from a neurodegenerative disease or disorder. As used herein, the term “neuroprotection” is synonymous with “protection of neurons”.

As used herein, promotion of neuronal survival is considered equivalent to neuroprotection

The term “regeneration” as used herein refers to enabling an increase in the activity of an injured or disabled cell, or a cell having below normal activity relative to the natural activity of a corresponding healthy cell. Such a cell may be a neuron. In some embodiments provided herein, “regeneration” refers to the regeneration of neurons in a patient having a neurodegenerative disease or disorder.

Thus, in some embodiments “neuroregeneration” refers to the regeneration of neurons in a patient having a neurodegenerative disease or disorder. In some embodiments, “neuroregeneration refers to the process of reversing either the loss of neuronal cells, or the loss of neuronal function occurring as a result of exposure to a neurotoxin or resulting from a neurodegenerative disease.

Neurorestoration shall be defined to be equivalent to neuroregeneration.

The term “neuronal function” as used herein refers to the capability of a neuron to synthesize, store, release, transport and respond to a neurotransmitter. Thus, changes in expression or integrity of certain components of neurons, including but not limited to receptors transporters, vesicles, cell bodies, axons or dendrites may affect neuronal function.

Neurotransmitters shall be defined as diffusible molecules released by neurons that either stimulate or inhibit neuronal activity.

The expression “low dose” as used herein refers to a dose of a compound or drug, e.g. bexarotene, not greater than 75 mg per day or 1 mg per kg body weight per day for a human patient. To obtain the desired effect of the compound or drug, at least when bexarotene is used, the dose shall be at least 0.05 mg per day or 0.0006 mg per kg body weight per day for a human patient. In some embodiments, the “low dose” may thus be a dose of from about 0.05 mg per day to about 75 mg per day, or from about 0.0006 mg per kg body weight per day to about 1 mg per kg body weight per day. The low dose may be give as one single daily dose or as a series of several doses or as a continuous infusion with a total daily dose of from about 0.05 mg to about 75 mg, or from about 0.0006 mg per kg body weight per day to about 1 mg per kg body weight per day. It is also possible to give the total low daily dose through at least two different routes of administration. Without being bound by any particular theory, it may be possible to use a low dose of bexarotene as provided herein based on the surprising finding that bexarotene is more than 10-fold more potent in stimulating Nurr-1-RXR heterodimers than RXR-RXR homodimers. Hence, for clinical applications that depend on Nurr-1 stimulation, bexarotene can be used in much lower and much more tolerated doses than are used in anti-cancer therapy. This is supported by studies in an animal model of PD that show neuroprotective and neuroregenerative effects of very low doses of bexarotene, as shown further below. Bexarotene is a RXR agonist that acts through the homodimer RXR-RXR to produce clinically used anti-cancer effects. It has been found that bexarotene given at a low dose is well tolerated yet effective. It has further been found that bexarotene can be used to slow down, stop or even restore neurodegeneration, which is further discussed and demonstrated below.

As used herein, “pharmaceutically acceptable salt” refers to a salt of a compound that does not per se abrogate the biological activity and properties of the compound. Pharmaceutical salts can be obtained by reaction of a compound disclosed herein with a base. Base-formed salts include, without limitation, ammonium salt (NH₄⁺); alkali metal, such as, without limitation, sodium or potassium, salts; alkaline earth, such as, without limitation, calcium or magnesium, salts; salts of organic bases such as, without limitation, dicyclohexylamine, N-methyl-D-glucamine, tris(hydroxymethyl)methylamine; and salts with the amino group of amino acids such as, without limitation, arginine and lysine.

Pharmaceutically acceptable solvates and hydrates are complexes of a compound with one or more solvent of water molecules, or 1 to about 100, or 1 to about 10, or one to about 2, 3 or 4, solvent or water molecules.

As used herein, to “modulate” the activity of a receptor means either to activate it, i.e., to increase its cellular function over the base level measured in the particular environment in which it is found, or deactivate it, i.e., decrease its cellular function to less than the measured base level in the environment in which it is found and/or render it unable to perform its cellular function at all, even in the presence of a natural binding partner. A natural binding partner is an endogenous molecule that is an agonist for the receptor.

An “agonist” is defined as a compound that increases the basal activity of a receptor (i.e. signal transduction mediated by the receptor).

As used herein, “partial agonist” refers to a compound that has an affinity for a receptor but, unlike an agonist, when bound to the receptor elicits only a fractional degree of the pharmacological response normally associated with the receptor even if a large number of receptors are occupied by the compound.

An “inverse agonist” is defined as a compound, which reduces, or suppresses the basal activity of a receptor, such that the compound is not technically an antagonist but, rather, is an agonist with negative intrinsic activity.

As used herein, “antagonist” refers to a compound that binds to a receptor to form a complex that does not give rise to any response, as if the receptor was unoccupied. An antagonist attenuates the action of an agonist on a receptor. An antagonist may bind reversibly or irreversibly, effectively eliminating the activity of the receptor permanently or at least until the antagonist is metabolized or dissociates or is otherwise removed by a physical or biological process.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional such as an M.D. or a D.V.M. to attempt to cure, or at least ameliorate the effects of, a particular disease or disorder or to prevent the disease or disorder from occurring in the first place.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

A “receptor” is intended to include any molecule present inside or on the surface of a cell that may affect cellular physiology when it is inhibited or stimulated by a ligand. Typically, a receptor comprises an extracellular domain with ligand-binding properties, a transmembrane domain that anchors the receptor in the cell membrane, and a cytoplasmic domain that generates a cellular signal in response to ligand binding (“signal transduction”). A receptor also includes any molecule having the characteristic structure of a receptor, but with no identifiable ligand. In addition, a receptor includes a truncated, modified, mutated receptor, or any molecule comprising partial or all of the sequences of a receptor.

“Ligand” is intended to include any substance that interacts with a receptor.

The “Nurr1 receptor” is defined as a receptor having an activity corresponding to the activity of the Nurr1 receptor subtype characterized through molecular cloning and pharmacology.

As used herein, “coadministration” of pharmacologically active compounds refers to the delivery of two or more separate chemical entities, whether in vitro or in vivo. Coadministration means the simultaneous delivery of separate agents; the simultaneous delivery of a mixture of agents; as well as the delivery of one agent followed by delivery of a second agent or additional agents. Agents that are coadministered are typically intended to work in conjunction with each other.

The term “an effective amount” as used herein means an amount of active compound or pharmaceutical agent that elicits the biological or medicinal response in a tissue, system, animal or human that is being sought by a researcher, veterinarian, medical doctor or other clinician, which includes alleviation or palliation of the symptoms of the disease being treated.

### Compounds

The compound as provided herein is bexarotene, the compound according to formula I (also known under the tradename Targretin and as LGD1069),

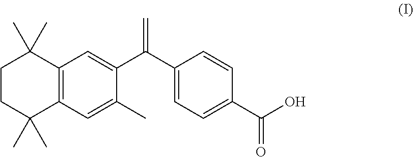 [SMILES: C=C(c1ccc(C(=O)O)cc1)c1cc2c(cc1C)C(C)(C)CCC2(C)C]

or a pharmaceutically acceptable salt, solvate, polymorph or hydrate thereof.

In some embodiments, bexarotene or a pharmaceutically acceptable salt, solvate, polymorph or hydrate thereof is coadministered with at least one other pharmacologically active compound.

As disclosed herein bexarotene or a pharmaceutically acceptable salt, solvate, polymorph or hydrate thereof or a pharmaceutical composition comprising any of these is to be administered in a low dose in any known or/and conventional administration route. Examples of suitable routes of administration include oral, rectal, transmucosal (including sublingual and buccal), topical, transdermal or intestinal administration; parenteral delivery, including intramuscular, subcutaneous, intravenous, intramedullary injections, as well as intrathecal, direct intracerebroventricular injection, direct injections to the human brain, direct intraventricular, intraperitoneal, intranasal, or intraocular injections. The compounds can also be administered in sustained or controlled release dosage forms, including nanoparticles, depot injections, osmotic pumps, electronic pumps, pills, transdermal (including electrotransport) patches, and the like, for prolonged and/or timed, pulsed administration at a predetermined rate. Sustained or controlled release dosage forms may be used to increase CNS exposure and minimize systemic exposure. It is also possible to combine at least two different routes of administration.

In some embodiments the compound is to be administered to a patient having a neurodegenerative disease or disorder in a dose of at least 0.05 mg up to, and including, 75 mg per day.

In some embodiments the compound is to be administered to a patient having a neurodegenerative disease or disorder in a dose of up to, and including, 70 mg per day. Some of these embodiments may relate to oral administration.

In some embodiments the compound is to be administered to a patient having a neurodegenerative disease or disorder in a dose of up to, and including, 65 mg per day.

In some embodiments the compound is to be administered to a patient having a neurodegenerative disease or disorder in a dose of up to, and including, 50 mg per day.

In some embodiments the compound is to be administered to a patient having a neurodegenerative disease or disorder in a dose of up to, and including, 20 mg per day. Some of these embodiments may relate to intracerebroventricular administration.

In some embodiments the compound is to be administered to a patient having a neurodegenerative disease or disorder in a dose of up to, and including, 15 mg per day. Some of these embodiments may relate to intracerebroventricular administration.

The lower limit of the dose range may be 0.05 mg per day, as indicated further above.

In some embodiments, the lower limit of the dose range may be 0.08 mg per day, with the upper limit according to any of the alternative embodiments given above. Some of these embodiments may relate to intracerebroventricular administration.

In some embodiments, the lower limit of the dose range may be 0.1 mg per day, with the upper limit according to any of the alternative embodiments given above.

In some embodiments, the lower limit of the dose range may be 0.5 mg per day, with the upper limit according to any of the alternative embodiments given above.

In some embodiments, the lower limit of the dose range may be 1 mg per day, with the upper limit according to any of the alternative embodiments given above.

In some embodiments, the lower limit of the dose range may be 5 mg per day, with the upper limit according to any of the alternative embodiments given above. Some of these embodiments may relate to oral administration.

In some embodiments, the lower limit of the dose range may be 3 mg per day, with the upper limit according to any of the alternative embodiments given above. Some of these embodiments may relate to subcutaneous administration.

In some embodiments, the lower limit of the dose range may be 12 mg per day, with the upper limit according to any of the alternative embodiments given above. Some of these embodiments may relate to oral administration.

In some embodiments the dose range may be selected from the group consisting of:

from 0.05 mg up to, and including, 75 mg per day,
from 0.05 mg up to, and including, 70 mg per day,
from 0.05 mg up to, and including, 65 mg per day,
from 0.05 mg up to, and including, 60 mg per day,
from 0.05 mg up to, and including, 59 mg per day,
from 0.05 mg up to, and including, 50 mg per day,
from 0.05 mg up to, and including, 20 mg per day,
from 0.05 mg up to, and including, 18 mg per day,
from 0.05 mg up to, and including, 15 mg per day,
from 0.05 mg up to, and including, 10 mg per day,
from 0.05 mg up to, and including, 5 mg per day,
from 0.05 mg up to, and including, 3 mg per day,
from 0.08 mg up to, and including, 75 mg per day,
from 0.08 mg up to, and including, 70 mg per day,
from 0.08 mg up to, and including, 65 mg per day,
from 0.08 mg up to, and including, 60 mg per day,
from 0.08 mg up to, and including, 59 mg per day,
from 0.08 mg up to, and including, 50 mg per day,
from 0.08 mg up to, and including, 20 mg per day,
from 0.08 mg up to, and including, 18 mg per day,
from 0.08 mg up to, and including, 15 mg per day,
from 0.08 mg up to, and including, 10 mg per day,
from 0.08 mg up to, and including, 5 mg per day,
from 0.08 mg up to, and including, 3 mg per day,
from 0.1 mg up to, and including, 75 mg per day,
from 0.1 mg up to, and including, 70 mg per day,
from 0.1 mg up to, and including, 65 mg per day,
from 0.1 mg up to, and including, 60 mg per day,
from 0.1 mg up to, and including, 59 mg per day,
from 0.1 mg up to, and including, 50 mg per day,
from 0.1 mg up to, and including, 20 mg per day,
from 0.1 mg up to, and including, 18 mg per day,
from 0.1 mg up to, and including, 15 mg per day,
from 0.1 mg up to, and including, 10 mg per day,
from 0.1 mg up to, and including, 5 mg per day,
from 0.1 mg up to, and including, 3 mg per day,
from 0.5 mg up to, and including, 75 mg per day,
from 0.5 mg up to, and including, 70 mg per day,
from 0.5 mg up to, and including, 65 mg per day,
from 0.5 mg up to, and including, 60 mg per day,
from 0.5 mg up to, and including, 59 mg per day,
from 0.5 mg up to, and including, 50 mg per day,
from 0.5 mg up to, and including, 20 mg per day,
from 0.5 mg up to, and including, 18 mg per day,
from 0.5 mg up to, and including, 15 mg per day,
from 0.5 mg up to, and including, 10 mg per day,
from 0.5 mg up to, and including, 5 mg per day,
from 0.5 mg up to, and including, 3 mg per day,
from 1 mg up to, and including, 75 mg per day,
from 1 mg up to, and including, 70 mg per day,
from 1 mg up to, and including, 65 mg per day,
from 1 mg up to, and including, 60 mg per day,
from 1 mg up to, and including, 59 mg per day,
from 1 mg up to, and including, 50 mg per day,
from 1 mg up to, and including, 20 mg per day,
from 1 mg up to, and including, 18 mg per day,
from 1 mg up to, and including, 15 mg per day,
from 1 mg up to, and including, 10 mg per day,
from 1 mg up to, and including, 5 mg per day,
from 1 mg up to, and including, 3 mg per day,
from 3 mg up to, and including, 75 mg per day,
from 3 mg up to, and including, 70 mg per day,
from 3 mg up to, and including, 65 mg per day,
from 3 mg up to, and including, 60 mg per day,
from 3 mg up to, and including, 59 mg per day,
from 3 mg up to, and including, 50 mg per day,
from 3 mg up to, and including, 20 mg per day,
from 3 mg up to, and including, 18 mg per day,
from 3 mg up to, and including, 15 mg per day,
from 3 mg up to, and including, 10 mg per day,
from 3 mg up to, and including, 5 mg per day,
from 5 mg up to, and including, 75 mg per day,
from 5 mg up to, and including, 70 mg per day,
from 5 mg up to, and including, 65 mg per day,
from 5 mg up to, and including, 60 mg per day,
from 5 mg up to, and including, 59 mg per day,
from 5 mg up to, and including, 50 mg per day,
from 5 mg up to, and including, 20 mg per day,
from 5 mg up to, and including, 18 mg per day,
from 5 mg up to, and including, 15 mg per day,
from 5 mg up to, and including, 10 mg per day,
from 10 mg up to, and including, 75 mg per day,
from 10 mg up to, and including, 70 mg per day,
from 10 mg up to, and including, 65 mg per day,
from 10 mg up to, and including, 60 mg per day,
from 10 mg up to, and including, 59 mg per day,
from 10 mg up to, and including, 50 mg per day,
from 10 mg up to, and including, 20 mg per day,
from 10 mg up to, and including, 18 mg per day,
from 10 mg up to, and including, 15 mg per day,
from 12 mg up to, and including, 75 mg per day,
from 12 mg up to, and including, 70 mg per day,
from 12 mg up to, and including, 65 mg per day,
from 12 mg up to, and including, 60 mg per day,
from 12 mg up to, and including, 59 mg per day,
from 12 mg up to, and including, 50 mg per day,
from 12 mg up to, and including, 20 mg per day, and
from 12 mg up to, and including, 18 mg per day,
from 12 mg up to, and including, 15 mg per day.

The doses given above are daily total doses estimated for an average sized human adult, weighing approximately 80 kg and with a height of approximately 180 cm. Doses may also be given as mg/kg/day or as mg/m²/day, wherein the kg is the weight of the subject, for example a human, to which the drug is to be administered and the m² is the area of the skin of the patient to which the drug is to be administered. The doses in mg/kg/day were calculated by dividing the dose in mg/day by 80 kg. The doses in mg/m²/day were calculated by multiplying doses in mg/day by the Km value for an 80 kg, 180 cm individual. The Km factor, bodyweight (kg) divided by body surface area (BSA in m²), is used to convert the mg/kg dose used in a study to an mg/m2 dose. Formulas for determining Km and BSA were from Reagan-Shaw et al. (Reagan-Shaw et al., 2008). The doses given in these ways above corresponding to the doses given above may be found below:

| mg/day | mg/kg/day | mg/m²/day |
|---|---|---|
| 75 | 0.94 | 38 |
| 70 | 0.88 | 35 |
| 65 | 0.81 | 33 |
| 60 | 0.75 | 30 |
| 59 | 0.74 | 29.6 |
| 50 | 0.63 | 25 |
| 20 | 0.25 | 10 |
| 18 | 0.23 | 9 |
| 15 | 0.19 | 7.5 |
| 12 | 0.15 | 6 |
| 10 | 0.13 | 5 |
| 7.5 | 0.09 | 3.8 |
| 5 | 0.063 | 2.5 |
| 3 | 0.038 | 1.5 |
| 1 | 0.013 | 0.0.5 |
| 0.5 | 0.0063 | 0.25 |
| 0.1 | 0.0013 | 0.05 |
| 0.08 | 0.001 | 0.04 |
| 0.05 | 0.0006 | 0.025 |

The drug may alternatively to the mg/day doses given above, also be used, administered or prescribed in mg/kg/day. Upper limits of dose ranges given in mg/kg/day may be selected from the group consisting of 1, 0.9, 0.8, 0.7, 0.6, 0.3, 0.2, 0.1, 0.06 and 0.04. Lower limits of dose ranges given in mg/kg/day may be selected from the group consisting of 0.0006, 0.001, 0.006, 0.01, 0.04, 0.06, 0.1 and 0.2. Since the effective dose may vary depending on the route of administration used, some doses that constitute upper limits for some routs of administration may constitute lower limits for other routes of administration.

Alternatively, the drug may be used, administered or prescribed in mg/m²/day dose. It is well known to the skilled person how to convert a dose given in mg/kg/day to mg/m²/day. It is also possible to use the conversion help as described (Reagan-Shaw et al., 2008).

The doses given above are daily doses or doses per day (known as QD dosing), i.e. the total amount in mg, mg/kg or mg/m², respectively, to be given per every 24 hours. However, the total amount given in each administration may vary. For example, the total daily amounts given above may be given once daily, or divided into one, two or three daily administrations. Furthermore, in some embodiments it may not be necessary to administer the drug every day. For example, the drug may then be administered once every second, third or fourth day, or once weekly. The amount to be administered at every such occasion is then calculated to that the average total daily amount is as mentioned above; for example, the amounts specified above may be doubled when the drug is administered once every second day.

In some embodiments the compound may be administered non-orally. Non-oral administration means that the treatment may be safer and more effective compared to oral administration may be more easily tolerated by the subject since it is possible to use a lower total dose of bexarotene or the pharmaceutically acceptable salt, solvate, polymorph or hydrate thereof, that the effects on liver function are reduced because the maximum concentrations of drug the liver is exposed to are reduced, and that the distribution of bexarotene in the body is altered such that a greater proportion of the administered dose reaches the brain compared with the periphery, thereby reducing many side-effects earlier associated with bexarotene.

In some embodiments the compound may be administered intracerebroventricularly (i.c.v.). I.c.v. administration means that the treatment may be safer and more effective compared to oral administration since it is possible to use a much lower total dose of bexarotene or the pharmaceutically acceptable salt, solvate, polymorph or hydrate thereof and that the distribution of bexarotene in the body is altered such that the vast majority of the administered dose is in the brain but very little gets into the periphery, thereby avoiding many side-effects earlier associated with bexarotene. This also improves the efficacy of bexarotene, since high concentrations are delivered into the brain with minimal concentrations in the periphery.

Some embodiments wherein intracerebroventricular administration may be preferred may relate to treatment of Parkinson's disease.

Other embodiments wherein intracerebroventricular administration may be preferred may relate to treatment of Alzheimer's disease, Huntington's disease, frontotemporal lobar degeneration associated with protein TDP-43 (FTLD-TDP), Dementia with Lewy bodies (DLB), vascular dementia and/or Amyotrophic lateral sclerosis (ALS).

In some embodiment subcutaneous administration may be preferred. Some of these embodiments may relate to treatment of Parkinson's disease.

Other embodiments wherein subcutaneous administration may be preferred may relate to treatment of Alzheimer's disease, Huntington's disease, frontotemporal lobar degeneration associated with protein TDP-43 (FTLD-TDP), Dementia with Lewy bodies (DLB), vascular dementia and/or Amyotrophic lateral sclerosis (ALS).

In some embodiment topical or transdermal administration may be preferred. Some of these embodiments may relate to treatment of Parkinson's disease.

Other embodiments wherein topical or transdermal administration may be preferred may relate to treatment of Alzheimer's disease, Huntington's disease, frontotemporal lobar degeneration associated with protein TDP-43 (FTLD-TDP), Dementia with Lewy bodies (DLB), vascular dementia and/or Amyotrophic lateral sclerosis (ALS).

In the context of the present disclosure it has been shown that it is possible to administer bexarotene or a pharmaceutically acceptable salt, solvate, polymorph or hydrate thereof or a pharmaceutical composition comprising any of these in a low dose, as defined above, thereby minimizing the deleterious side effects but still obtaining the desired therapeutic effect.

Such deleterious side effects that may be decreased or minimized according to the present disclosure include, but are not limited to i.a. hyperlipidaemia, acute pancreatitis, liver function test (LFT) abnormalities and in particular LFT elevations, thyroid function test alterations and most often elevations in serum triglycerides and serum cholesterol, reductions in thyroid hormone (total thyroxine, T₄) and thyroid-stimulating hormone (TSH), leucopenia, anaemia, lens opacities, hypoglycaemia in patients with diabetes mellitus, bleeding, hemorrhage, and coagulopathy, dyspnea, nausea, neuropathic pain, edema, anorexia, asthenia, fatigue, leucopenia, pancreatitis and dehydration and photosensitivity.

In some embodiments the negative side effect to be minimized is hyperlipidaemia.

In some embodiments the negative side effect to be minimized is hypertriglyceradaemia.

In some embodiments the negative side effect to be minimized is hypercholesterolaemia.

In some embodiments the negative side effect to be minimized is the reduction of T₄ levels.

In some embodiments the negative side effect to be minimized is the reduction of TSH levels.

When administered to a subject or a patient, bexarotene or a pharmaceutically acceptable salt, solvate, polymorph or hydrate thereof or a pharmaceutical composition comprising any of these may lead to regeneration of the function of dopaminergic neurons.

According to the present disclosure it may thus be possible to restore function to dopaminergic neurons that have lost function due to a neurodegenerative disorder or a neurodegenerative condition. Possible ways of measuring restoration of the function of dopaminergic neurons in humans afflicted with a neurodegenerative disorder or a neurodegenerative condition include, but are not limited to using PET (positron emission tomography) to measure dopamine turnover, or DAT (dopamine transporter) activity, or neuroinflammatory markers.

In some embodiments the dopaminergic neurons have lost their function partially due to Parkinson's disease. The fact that the function of dopaminergic neurons may be regenerated means that it may be possible to reverse the progression of the disease. This is not possible with compounds that only slow down the progression of the disease. Possible ways of measuring the effect on neurodegeneration or neuroregeneration include, but are not limited to using PET (positron emission tomography) to measure dopamine turnover, or DAT (dopamine transporter) activity, or neuroinflammatory markers. Other ways of measuring the effect on neurodegeneration or neuroregeneration could be to measure the symptoms caused by neurodegeneration. For example one may use unified Parkinson's disease rating scale (UPDRS).

Bexarotene or a pharmaceutically acceptable salt, solvate, polymorph or hydrate thereof or a pharmaceutical composition comprising any of these may therefore be used in treatment of a disease or disorder that benefits from regeneration of dopaminergic neurons.

Such diseases or disorders that benefits from regeneration of dopaminergic neurons may be diseases or disorders associated with a Nurr1 receptor.

In some embodiments the compound as provided herein or pharmaceutically acceptable salt, solvate, polymorph or hydrate thereof leads to an increased activity of a Nurr1 receptor upon administration to the subject.

In some embodiments the activity of the Nurr1 receptor is a signaling activity of a receptor complex including the Nurr1 receptor.

In some embodiments the activity of the Nurr1 receptor is associated with Nurr1 receptor activation.

In some embodiments the Nurr1 receptor is located in the subject's central nervous system.

The compound may form part of a pharmaceutical composition. The term “pharmaceutical composition” refers to a mixture of a compound disclosed herein with other chemical components, such as diluents or carriers. The introduction of the compound into a pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

The pharmaceutical compositions of bexarotene may be manufactured in a manner that is itself known, e.g., by means of conventional mixing, dissolving, granulating, dragee-making, levigating, emulsifying, encapsulating, entrapping or tabletting processes.

Pharmaceutical compositions of bexarotene for use as described herein may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations (for example, liposomes), may be utilized.

For transmucosal administration, penetrants appropriate to the barrier to be permeated may be used in the formulation.

For transdermal administration, the composition may be formulated as a gel.

Pharmaceutical formulations for parenteral administration, e.g., by bolus injection or continuous infusion, include aqueous solutions of the active compounds in water-soluble form. Additionally, suspensions of the active compounds may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For oral administration, bexarotene can be formulated readily by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

Buccal administration refers to placing a tablet between the teeth and the mucous membranes of the cheek any composition suitable therefor is thus contemplated. The compositions may for example take the form of tablets or lozenges formulated in conventional manner.

For administration by inhalation, bexarotene, for use as described herein, is conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Further disclosed herein are various pharmaceutical compositions well known in the pharmaceutical art for uses that include intraocular, intranasal, and intraauricular delivery. Suitable penetrants for these uses are generally known in the art. Pharmaceutical compositions for intraocular delivery include aqueous ophthalmic solutions of the active compounds in water-soluble form, such as eyedrops, or in gellan gum (Shedden et al., Clin. Ther., 23(3):440-50 (2001)) or hydrogels (Mayer et al., Ophthalmologica, 210(2):101-3 (1996)); ophthalmic ointments; ophthalmic suspensions, such as microparticulates, drug-containing small polymeric particles that are suspended in a liquid carrier medium (Joshi, A., J. Ocul. Pharmacol., 10(1):29-45 (1994)), lipid-soluble formulations (Alm et al., Prog. Clin. Biol. Res., 312:447-58 (1989)), and microspheres (Mordenti, Toxicol. Sci., 52(1):101-6 (1999)); and ocular inserts. All of the above-mentioned references, are incorporated herein by reference in their entireties. Such suitable pharmaceutical formulations are most often and preferably formulated to be sterile, isotonic and buffered for stability and comfort. Pharmaceutical compositions for intranasal delivery may also include drops and sprays often prepared to simulate in many respects nasal secretions to ensure maintenance of normal ciliary action. As disclosed in Remington's Pharmaceutical Sciences, 18th Ed., Mack Publishing Co., Easton, Pa. (1990), which is incorporated herein by reference in its entirety, and well-known to those skilled in the art, suitable formulations are most often and preferably isotonic, slightly buffered to maintain a pH of 5.5 to 6.5, and most often and preferably include antimicrobial preservatives and appropriate drug stabilizers. Pharmaceutical formulations for intraauricular delivery include suspensions and ointments for topical application in the ear. Common solvents for such aural formulations include glycerin and water.

Bexarotene may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For hydrophobic compounds, a suitable pharmaceutical carrier may be a co-solvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Agents intended to be administered intracellularly may be administered using techniques well known to those of ordinary skill in the art. For example, such agents may be encapsulated into liposomes. All molecules present in an aqueous solution at the time of liposome formation are incorporated into the aqueous interior. The liposomal contents are both protected from the external micro-environment and, because liposomes fuse with cell membranes, are efficiently delivered into the cell cytoplasm. The liposome may be coated with a tissue-specific antibody. The liposomes will be targeted to and taken up selectively by the desired organ. Alternatively, small hydrophobic organic molecules may be directly administered intracellularly.

Additional therapeutic or diagnostic agents may be incorporated into the pharmaceutical compositions. Alternatively or additionally, pharmaceutical compositions may be combined with other compositions that contain other therapeutic or diagnostic agents.

### Methods of Administration

Bexarotene may be administered to the patient by any suitable means. Non-limiting examples of methods of administration include, among others, (a) administration though oral pathways, which administration includes administration in capsule, tablet, granule, spray, syrup, or other such forms; (b) administration through non-oral pathways such as rectal, vaginal, intraurethral, intraocular, intranasal, intracerebroventricular or intraauricular, which administration includes administration as an aqueous suspension, an oily preparation or the like or as a drip, spray, suppository, salve, ointment or the like; (c) administration via injection, subcutaneously, intraperitoneally, intravenously, intramuscularly, transdermally, intraorbitally, intracapsularly, intraspinally, intrasternally, intracranially, intracerebroventricularly or the like, including infusion pump delivery; (d) administration locally such as by injection directly in the renal or cardiac area, e.g., by depot implantation; (e) administration topically; as deemed appropriate by those of skill in the art for bringing the compound disclosed herein into contact with living tissue as well as f) administration as aerosols via inhalation.

Pharmaceutical compositions of bexarotene suitable for administration include compositions where the active ingredients are contained in an amount effective to achieve its intended purpose. However, as indicated above, the compound is to be administered in a low dose. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. More specifically, in the context of the present disclosure, a therapeutically effective amount means an amount of compound effective to prevent, alleviate, ameliorate or modify a disease or prolong the survival of the subject being treated. Determination of a therapeutically effective amount is well within the capability of those skilled in the art, especially in light of the detailed disclosure provided herein.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved or adverse side effects disappear. Alternatively dosages may be based and calculated upon the surface area of the patient, as understood by those of skill in the art.

The exact formulation, route of administration and dosage for the pharmaceutical compositions disclosed herein can be chosen by the individual physician in view of the patient's condition. (See e.g., Fingl et al. 1975, in “The Pharmacological Basis of Therapeutics”, which is hereby incorporated herein by reference in its entirety, with particular reference to Ch. 1, p. 1). The dosage may be a single one or a series of two or more given in the course of one or more days, as is needed by the patient.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

In some embodiments, the compounds will be administered for a period of continuous therapy, for example for a week or more, or for months or years.

The amount of composition administered may be dependent on the subject being treated, on the subject's weight, the severity of the affliction, the manner of administration and the judgment of the prescribing physician.

The compositions of bexarotene may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

Further details are provided in the following examples, which are not in any way intended to limit the scope of the invention as claimed.

### BRIEF DESCRIPTION OF THE DRAWINGS

In the following examples reference is made to the appended drawings which illustrate the following.

FIG. 1: discloses Bioluminescence Resonance Energy Transfer (BRET) constructs, where receptors are drawn with the amino-terminus on the left. Vertical lines denotes Green Fluorescent Protein (GFP2). A grid denotes Renilla luciferase (Rluc).

FIG. 2 discloses pharmacological profiling in BRET. Pairs of receptors, one tagged with Luc, one with GFP, were co-expressed, except for receptors labeled DT which were fused to both tags. BRET assays were performed using the indicated concentrations of ligands. Information about each compound is found in Table 1.

FIG. 3 illustrates bexarotene activity through interaction with a specific consensus sequence in the promoters of Nurr1 target genes, known as the nerve growth factor-induced clone B response element (also known as the NGFI-B response element or NBRE) enhancer driven luciferase reporter assays.

FIG. 4 illustrates neuroprotective effects of bexarotene in 6 hydroxydopamine (6-OHDA) treated rats.

FIG. 5 shows the pharmacokinetics in brain and plasma of bexarotene administered orally. Male Sprague-Dawley rats received once daily oral doses of 1 or 10 mg/kg/day bexarotene. Prior to the 5th dose, plasma and brain samples were analyzed to get T=0 (time) values. After the 5th dose, plasma and brain samples were obtained at were obtained at the indicated time intervals and analyzed for bexarotene concentrations. For the 1 mg/kg dose, the brain and plasma concentrations at T=0 and 24 hrs (not shown) were below the detection limit and were assigned values of 0.5 ng/ml (50% of the analytical detection limit) to permit estimation of AUC₀₋₂₄.

FIG. 6 displays the motor performance of sham (all treatments combined) and 6-hydroxydopamine animals treated with vehicle (Veh), or bexarotene starting 72 hours following 6OHDA infusion (bex(72)). Panels A and B show the start latency and time required to traverse the challenging beam, respectively. Panels C and D show trial time and rpm achieved on the rotorod, respectively. Panel E shows distance traveled during a 15 min spontaneous locomotor session. For each of these measures of motoric ability, 6OHDA treatment statistically impaired performance. In all cases, lesioned animals treated with bexarotene, 0.006 mg/kg/day administered i.c.v. (beginning 72 after lesion) were not impaired relative to Sham controls. Data were analyzed using one-way ANOVAs, followed by Bonferroni's multiple comparison post hoc analyses. * indicates a significant difference from sham treated animals, p<0.05. + indicates a significant difference from vehicle/6OHDA, p<0.05. N=7-9 animals per group.

FIG. 7 shows tyrosine hydroxylase immunofluorescence in the substantia nigra pars compacta (SNc) following sham- or 6OHDA-treatment. 6OHDA resulted in reduced cell counts in the SNc (Panel A), reduced mean cell size (Panel B), reduced mean pixel intensity of immunofluorescent pixels (Panel C), reduced percentage of the image that was immunopositive (Panel D) and reduced colocalization of TH positive cells with the general neuronal marker Neutrotrace (Panel E). Treatment with bexarotene, 0.006 mg/kg/day administered i.c.v. beginning 72 hours after 6OHDA lesion significantly improved cell counts, cell size and mean pixel intensity compared to vehicle treated subjects. Data were analyzed with one-way ANOVAs followed by Bonferroni's post hoc comparisons. * indicates a significant difference from Sham, p<0.05; + indicates a significant difference from vehicle/6OHDA, p<0.05.

FIG. 8 shows dopamine transporter (DAT), and vesicular monoamine transporter 2 (VMAT2), immunohistochemistry in the striatum following sham- or 6OHDA-treatment. 6OHDA reduced percentage of the image that was immunopositive (Panels A, C) and reduced mean pixel intensity of immunofluorescent pixels (Panels B, D). Treatment with bexarotene, 0.006 mg/kg/day administered i.c.v. beginning 72 hours after 6OHDA lesion significantly increased all measures. Data were analyzed with one-way ANOVAs followed by Bonferroni's post hoc comparisons. * indicates a significant difference from Sham, p<0.05; + indicates a significant difference from vehicle/6OHDA, p<0.05.

FIG. 9 displays the motor performance of sham (all treatments combined) and 6-hydroxydopamine animals treated with vehicle (Veh), or bexarotene (16 (16Bex), 4 (4Bex), 1 (1Bex), and 0.3 (0.3Bex) mM providing 1, 0.25, 0.0625 and 0.021 mg/kg/day) administered subcutaneously beginning 72 hours following 6OHDA infusion. Panels A and B show the start latency and time required to traverse the challenging beam, respectively. Panels C and D show trial time and rpm achieved on the rotorod, respectively. Panel E shows distance traveled during a 15 min spontaneous locomotor session. For each of these measures of motoric ability, 6OHDA treatment statistically impaired performance. In all cases, lesioned animals treated with bexarotene administered s.c. (sub-cutaneous) (beginning 72 after lesion) were not impaired relative to Sham controls. Data were analyzed using one-way ANOVAs, followed by Bonferroni's multiple comparison post hoc analyses. * indicates a significant difference from sham treated animals, p<0.05. + indicates a significant difference from vehicle/6OHDA, p<0.05. N=9-12 animals per group.

FIG. 10 shows tyrosine hydroxylase immunofluorescence in the SNc following sham- or 6OHDA-treatment. 6OHDA resulted in reduced mean pixel intensity (Panel A), reduced percentage of the image that was immunopositive (Panel B), reduced cell counts in the SNc (Panel C), and reduced co-localization of TH-positive cells with the general neuronal marker Neurotrace (Panel D). Treatment with bexarotene (16, 4, 1, and 0.3 mM providing 1, 0.25, 0.0625 and 0.021 mg/kg/day) administered s.c. beginning 72 hours after 6OHDA lesion significantly improved mean pixel intensity, percentage of the image that was immunopositive, cell counts, and percentage of TH-positive cells that co-localized with Neurotrace compared to vehicle treated subjects. Data were analyzed with one-way ANOVAs followed by Bonferroni's post hoc comparisons. * indicates a significant difference from Sham, p<0.05; + indicates a significant difference from vehicle/6OHDA, p<0.05.

FIG. 11 shows Ret-c (the co-receptor for the trophic factor GDNF (glial cell line-derived neurotrophic factor)) in the SNc following sham- or 6OHDA-treatment. 6OHDA resulted in reduced cell counts in the SNc (Panel A), reduced percentage of the image that was immunopositive (Panel B), and reduced mean pixel intensity of immunofluorescent pixels (Panel C). Treatment with bexarotene (Bex) (16 mM pump solution providing 1 mg/kg/day) administered s.c. beginning 72 hours after 6OHDA lesion significantly improved cell counts, percent immunopostive image, and mean pixel intensity compared to vehicle treated subjects. Data were analyzed with one-way ANOVAs followed by Bonferroni's post hoc comparisons. * indicates a significant difference from Sham, p<0.05; + indicates a significant difference from vehicle/6OHDA, p<0.05.

FIG. 12 shows bilateral lesions of the substantia nigra with 6-hydroxydopamine (Lesion/Veh) resulted in motor impairments in challenging beam (Panels A and B) and rotorod (Panels C and D) performance compared with Sham controls. It also resulted in impaired memory assessed with novel object recognition (Panel E) and augmented spontaneous head twitches (Panel F). In all cases, oral administration of bexarotene (Lesion/Drug, 1 or 3 but not 0.3 mg/kg/day orally for 28 days beginning 3 days post-lesion) normalized behavior disrupted by lesion. Data were analyzed with one-way ANOVAs followed by Bonferroni's post hoc comparisons. * indicates a significant difference from Sham, p<0.05; + indicates a significant difference from vehicle/6OHDA, p<0.05. N=10-15 animals per group.

FIG. 13 shows bilateral lesions of the substantia nigra with 6-hydroxydopamine resulted in a reduced number of tyrosine hydroxylase (TH) positive cells in the SNc (Panel A), reduced colocalization of TH with the neuronal marker Neurotrace (Panel B), reduced mean pixel intensity (Panel C), and reduced percentage of the image that was immunopositive (Panel D). Oral administration of bexarotene (1 or 3 but not 0.3 mg/kg/day for 28 days beginning 3 days after 6OHDA lesion) significantly improved the number of TH positive cells, mean pixel intensity, % immunopositive cells, and colocalization of TH and Neurotrace compared to vehicle treated subjects. Data were analyzed with one-way ANOVAs followed by Bonferroni's post hoc comparisons. * indicates a significant difference from Sham, p<0.05; + indicates a significant difference from vehicle/6OHDA, p<0.05.

FIG. 14 illustrates that bexarotene regenerates neurons. Compared with sham controls, animals treated with 6OHDA 31 days (Lesion/Veh) or 3 days (Day 3) prior to analysis displayed a reduced number of TH positive cells in the SNc (Panel A), reduced mean cell size (Panel B), and a reduced colocalization of TH with the neuronal marker Neurotrace. Treatment with bexarotene beginning 72 hours after 6OHDA lesion (Lesion/Bex(72)) for 28 days significantly improved the number of TH positive cells, cell size and colocalization of TH and Neurotrace. Notably, bexarotene treatment also significantly improved these measures when compared with animals sacrificed 3 days after lesion (i.e. at the start of bexarotene treatment).

FIG. 15 shows dose effect curve of bexarotene (denoted bexarotene in the figure) and BDNF (50 ng/ml) on TH positive neurons (a), on total TH neurite length (b), and TH positive neurons displaying neurites (c), when applied after a 24 h MPP+ injury (4 μM) expressed in percentage of control. (mean±s.e.m). *: p<0.05 groups vs MPP+; # MPP+ vs Control.

FIG. 16 shows representative pictures of the neurotrophic effect observed in FIG. 15.

FIG. 17 shows bexarotene effects on serum triglyceride and T4 levels. Rats were administered bexarotene either through continuous infusion of bexarotene solutions at the indicated concentrations through intracranial pumps (i.c.v., 0.1 mM, 0.3 mM and 1 mM correspond to 0.000625, 0.002, and 0.00625 mg/kg/day) for either 4 days (D4) or 8 days (D8), or as once daily oral (P.O.) doses at 1, 3, 10, 30 or 100 mg/kg/day for 5 days. At the end of the indicated dosing periods, blood was harvested, and the serum analyzed for triglyceride (FIG. 17A) and T4 levels (FIG. 17B) by IDEXX Corporation.

FIG. 18 shows the interspecies correlation of AUC with bexarotene dose. AUC values derived from PK experiments using oral doses of Bexarotene. Linear regression was fitted through x=0 and y=0. The correlation coefficient is excellent (r²>0.99). Thus one can extrapolate AUC between species. Data were taken from Targretin NDA #21055; targretin being the tradename of Bexarotene.

FIG. 19 shows that AUC is proportional to bexarotene dose in humans. AUC values derived from PK experiments using bexarotene dosed orally to human subjects. Linear regression was fitted through x=0 and y=0. The slopes (m1 and m2) were 1.735 and 2.030 for (A) and (B), respectively. Data taken from Miller et al., J. Clin. Oncol. 1997 (A) and Rizvi et al., Clin. Cancer Res., 1999 (B).

### EXAMPLES

### Example 1

### Screening of Test Compounds in an Assay Using Nurr1 Receptor BRET Assays

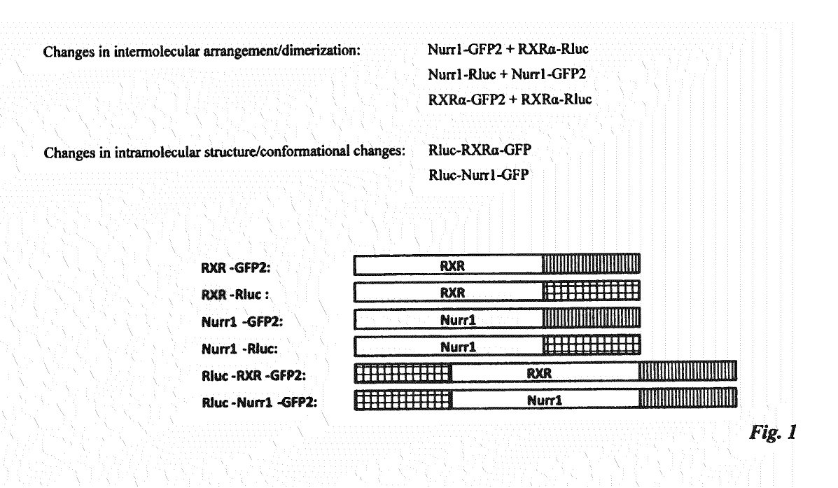

We have established intramolecular and intermolecular BRET (Bioluminescence Resonance Energy Transfer) assays of Nurr1 and RXR (Retinoic receptors such as, RXR-alpha, RXR-beta, and RXR-gamma) receptors by tagging each receptor with either Green Fluorescent Protein (GFP2) or Renilla luciferase (Rluc) or both tags together (see FIG. 1). BRET occurs only when the Rluc moiety is within 100 angstroms of the GFP moiety (Pfleger and Eidne, 2003), thus these assays enable us to test each receptor for ligand-induced rearrangement of its tertiary and quartenary structures as disclosed in FIG. 1. BRET assays were performed as described (Tan et al., 2007) in the following: HEK293T cells cultured in 10 cm² plates were transiently transfected with plasmid DNAs expressing a bioluminescence donor (1 μg plasmid DNA) expressing a receptor carboxy-terminally tagged with Renilla luciferase and a fluorescence acceptor (20 μg plasmid DNA) expressing a receptor amino-terminally tagged with GFP2. The receptors were Nurr1 and RXR, each was tagged with Rluc, GFP2, or both tags as indicated in FIG. 1. Two days after transfection, cells were harvested and resuspended in BRET buffer (PBS containing 0.1% D-glucose and 1 mM sodium pyruvate) to a concentration of 2×10⁶-4×10⁶ cells/mL depending on transfection efficiency. 50 μl of 3-fold concentrated ligand dilutions were dispensed into wells of white, flat-bottomed, 96-well plates (Costar; Corning Life Sciences, Acton, Mass.). Ligands were incubated for 20 to 30 min with 50 μl of cell suspension to stimulate the interaction of Receptor-Luc (bioluminescence donor) with Receptor-GFP2 (fluorescence acceptor). The BRET-2 signal was detected directly after injecting 50 μl/well of 15 μM coelenterazine 400A (DeepBlueC; PerkinElmer Life and Analytical Sciences) diluted in PBS using a Mithras LB 940 plate reader (Berthold Technologies, Bad Wildbad, Germany). After 1 s of plate-shaking, luminescence emissions for Renilla luciferase and GFP2 were recorded through BRET-optimized filters (luciferase peak 410 nm; GFP2 peak, 515 nm) for 1 to 2 s. The BRET-2 signal was calculated as the ratio between the luciferase and the GFP2 emission corrected by the background emission of non-transfected cells.

A collection of ligands with diverse chemical structures and diverse reported pharmacological profiles (see Table 1) in the BRET assays described above were profiled.

TABLE 1
Compound collection. Shown are some of the compounds profiled in these
studies along with their proposed pharmacologies and primary references.

| Name | Structure | Reported Pharmacology | Reference |
|---|---|---|---|
| 9-cis-Retinoic Acid | 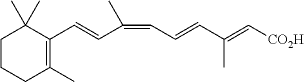 [SMILES: CC1=C(/C=C/C(C)=C\C=C\C(C)=C\C(=O)O)C(C)(C)CCC1] | Non-selective full retinoid agonist | |
| Bexarotene (Targretin/ LGD1069) | 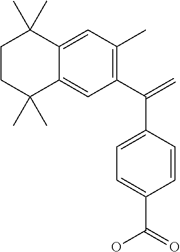 [SMILES: C=C(c1ccc(C(=O)O)cc1)c1cc2c(cc1C)C(C)(C)CCC2(C)C] | RXR selective agonist | Boehm et. al., J. Med. Chem., 1994. |
| LG100268 | 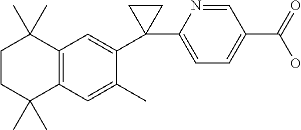 [SMILES: Cc1cc2c(cc1C1(c3ccc(C(=O)O)cn3)CC1)C(C)(C)CCC2(C)C] | RXR selective agonist | Boehm et. al., J. Med. Chem., 1995. |
| SR11237 | 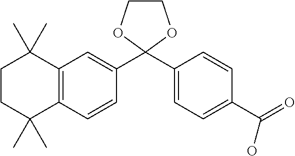 [SMILES: CC1(C)CCC(C)(C)c2cc(C3(c4ccc(C(=O)O)cc4)OCCO3)ccc21] | RXR selective agonist | Wallen-Mackenzie et al., Genes Dev., 2003. |
| XCT0135908 | 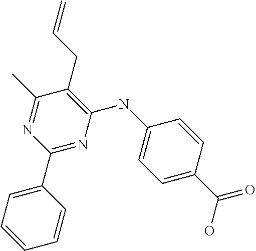 [SMILES: C=CCc1c(C)nc(-c2ccccc2)nc1Nc1ccc(C(=O)O)cc1] | RXR-Nurr1 heterodimer selective agonist | Wallen-Mackenzie et al., Genes Dev., 2003. |
| HX630 | 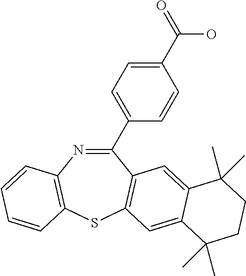 [SMILES: CC1(C)CCC(C)(C)c2cc3c(cc21)Sc1ccccc1N=C3c1ccc(C(=O)O)cc1] | RXR selective agonist/potentiator | compound 29 in Umemiya et al., J. Med. Chem., 1997. |
| PAO24 | 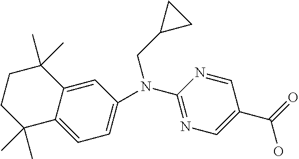 [SMILES: CC1(C)CCC(C)(C)c2cc(N(CC3CC3)c3ncc(C(=O)O)cn3)ccc21] | RXR selective agonist/potentiator | compound 10G in Ohta et at., J. Med. Chem., 2000. |
| AC-261066 | 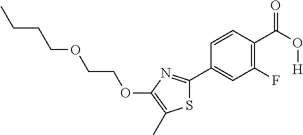 [SMILES: CCCCOCCOc1nc(-c2ccc(C(=O)O)c(F)c2)sc1C] | RARb2 selective agonist | Lund et al., J. Med. Chem., 2005. |
| AC271251 | 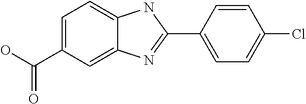 [SMILES: O=C(O)c1ccc2[nH]c(-c3ccc(Cl)cc3)nc2c1] | Putative Nurr1 agonist | compound 11 in Dubois et al., Chem. Med. Chem., 2006. |
| AC271252 | 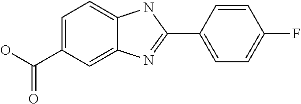 [SMILES: O=C(O)c1ccc2[nH]c(-c3ccc(F)cc3)nc2c1] | Putative Nurr1 agonist | compound 12 in Dubois et al., Chem. Med. Chem., 2006. |
| 6-MP | 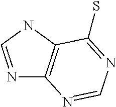 | Putative Nurr1 agonist/anticancer drug | Ordentlich et al., J. Biol. Chem., 2003. |
| 6-MP 2-deoxyribose | 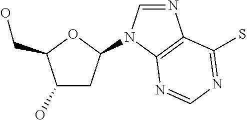 [SMILES: OC[C@H]1O[C@@H](n2cnc3c(S)ncnc32)C[C@@H]1O] | 6-MP active metabolite | Ordentlich et al., J. Biol. Chem., 2003. |
| 6-MP ribose | 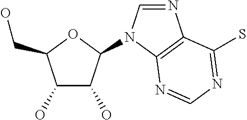 [SMILES: OC[C@H]1O[C@@H](n2cnc3c(S)ncnc32)[C@H](O)[C@@H]1O] | 6-MP active metabolite | Ordentlich et al., J. Biol. Chem., 2003. |

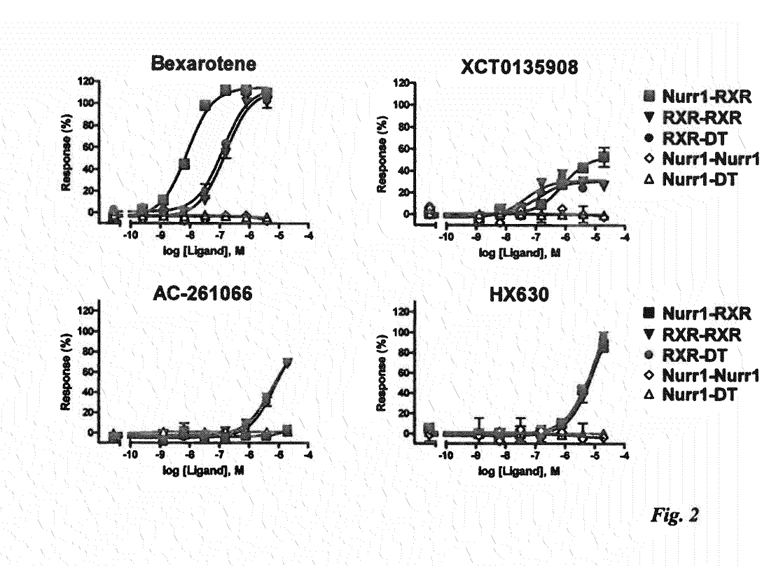

The results, which demonstrate the agonist activity of the compounds described herein, are presented in FIG. 2 and Table 2.

Ligands with diverse chemical and pharmacological profiles in BRET and observed clear examples of ligands with bias for and against formation of Nurr1-RXR heterodimers as is disclosed in FIG. 2 were profiled.

TABLE 2
Pharmacological profiling in BRET assays.

| | RXR-Luc & | | RXR-Luc & | | | |
|---|---|---|---|---|---|---|
| | Nurr1-GFP | | RXR-GFP | | Luc-RXR-GFP | |
| Ligand | pEC50 | Eff (%) | pEC50 | Eff (%) | pEC50 | Eff (%) |
| 9-cis-RA | 7.1 | 100 | 6.0 | 100 | 6.3 | 100 |
| Bexarotene | 7.9 | 105 | 6.7 | 94 | 6.9 | 93 |
| HX630 | 5.1 | 99 | 4.9 | 97 | 5.0 | 123 |
| PAO24 | 7.0 | 64 | 5.9 | 87 | 6.0 | 94 |
| XCT0135908 | 6.3 | 50 | 7.2 | 31 | 6.9 | 25 |
| AC261066 | — | NA | 5.2 | 65 | 5.1 | 85 |

AC-271251, AC-271252, 6-MP, 6-MP 2-deoxyribose, and 6-MP ribose were inactive in all assays. Potency is reported as the negative logarithm of the EC50 (pEC50).

Surprisingly the potent RXR-selective rexinoid bexarotene (Targretin) displayed the greatest selectivity and potency in promoting formation of Nurr1-RXR heterodimers (FIG. 2). The structurally related RXR agonists LG100268 and SR11237, showed similar selectivity to bexarotene in promoting formation of Nurr1-RXR heterodimers (not shown). XCT0135908, known as a selective Nurr1-RXR agonist (Wallen-Mackenzie et al, 2003), had greater maximum effect at Nurr1-RXR but was not more potent than at RXR-RXR. A structurally different rexinoid called HX630 (Umemiya et al, 1997) was equipotent at Nurr1-RXR and RXR-RXR, while the RARb2-selective compound AC-261066 (Lund et al, 2005) was active only at RXR-RXR. Surprisingly the putative Nurr1 agonists compounds II & 12 (Dubois et al, 2006) and 6-MP, 6-MP-ribose, and 6-MP-2-deoxyribose (Ordentlich et al, 2003) were inactive at all receptor combinations tested (data not shown).

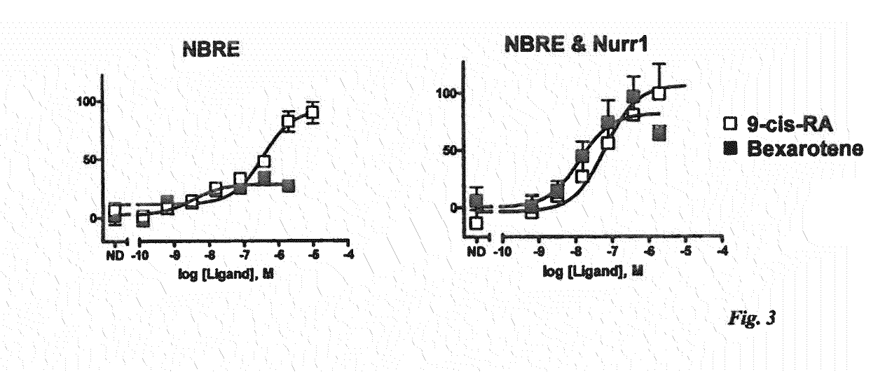

We have enabled assays to detect ligand-induced gene transcription (reporter gene assays) in order to confirm results obtained in BRET2 assays. Gene transcription is quantified by luciferase expression which is driven by response elements that respond to different nuclear receptors: the Retinoid X Receptor (RXR) response element RXRE, the Retinoic Acid Receptor (RAR) response element RARE, and the NGFI-B response element, NBRE, which is bound by Nurr1 monomers (Castro et al., 1999). We tested bexarotene in these assays and observed that its activity at RXRE and NBRE response elements, but not RARE response elements was increased substantially relative to the non-selective retinoid 9-cis retinoic acid when Nurr1 was co-expressed (Table 3 and FIG. 3) A similar pattern was seen with PA024, HX630 and XTC0135908.

TABLE 3
Pharmacological profiling in mammalian reporter gene transcription assays.
Potency is given in units of nanomolar. Maximum response is given as Eff % and is
normalized to the response of 9-cis retinoic acid.

| | | | pNBRE | | | | pRXRE | | | | pRARE | |
|---|---|---|---|---|---|---|---|---|---|---|---|---|
| | pNBRE | | & Nurr1 | | pRXRE | | & Nurr1 | | pRARE | | & Nurr1 | |
| Ligand | EC₅₀ (nM) | Eff % | EC₅₀ (nM) | Eff % | EC₅₀ (nM) | Eff % | EC₅₀ (nM) | Eff % | EC₅₀ (nM) | Eff % | EC_{50 (nM)} | Eff % |
| 9-cis-RA | 564 | 100 | 106 | 100 | 88 | 100 | 105 | 100 | 154 | 100 | 208 | 100 |
| Bexarotene | 20 | 28 | 17 | 83 | 144 | 44 | 41 | 65 | 30 | 19 | 26 | 23 |
| PA024 | 43 | 24 | 45 | 83 | 131 | 44 | 106 | 64 | 101 | 19 | 53 | 20 |
| HX630 | — | 16 | 18 | 41 | 664 | 26 | 2460 | 47 | — | 8 | — | 6 |
| XTC0135908 | — | 10 | 25 | 25 | — | 4 | 5321 | 49 | — | 3 | — | 0 |

### Example 2

### Bexarotene Protects Neurons

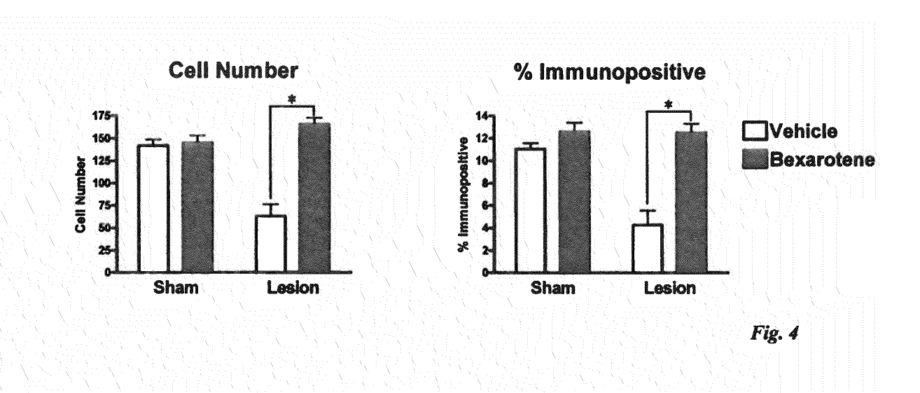

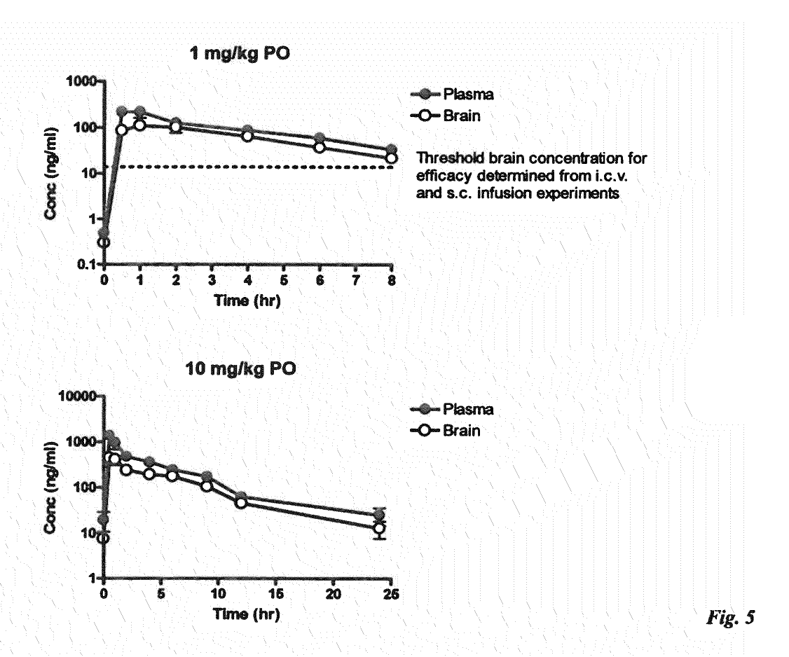

Based on the selective Nurr1-RXR profile of bexarotene, bexarotene was tested for the ability to protect against 6-OHDA (6-hydroxydopamine) induced neuronal loss in rodents. The results are shown in FIG. 4. Male Sprague-Dawley rats were implanted with bilateral guide cannulas 2 mm above the SNc. 5-7 days post-surgery, subjects received treatments which consisted of 3 daily microinjections of bexarotene (1 μL of 10 μM) or vehicle. 4 hrs following the second bexarotene treatment, subjects were injected with vehicle or 6-OHDA (4 μL, of 2 mg/ml) to induce loss of DA neurons. 48 hrs after the final microinjection, subjects were sacrificed. Their brains were fixed, sectioned through the SNc and labeled for tyrosine hydroxylase. Bilateral serial sections (3/side, −5.2 mm from bregma) were photographed and analyzed for the number of TH+ neurons and the % of the section that was immunopositive. 6-OHDA treatment (Lesion) produced a decrease in DA cell number and % of the section that was immunopositive relative to vehicle treated controls (Sham). As shown in FIG. 4, microinjected bexarotene completely prevented the loss of dopaminergic cells induced by 6-OHDA.

It could be concluded that there is a strong correlation between formation of Nurr1-RXR heterodimers in BRET and neuroprotection of DA (dopaminergic) neurons in models of PD (Parkinson's disease), with bexarotene being very effective in both.

### Example 3

### Bexarotene Concentrations in Plasma and Brain Administered Peripherally or Centrally

Bexarotene was administered peripherally by once daily oral dosing (P.O., QD), peripherally by continuous infusion sub-cutaneously (s.c.) (C.I. s.c.) using implanted pumps, and centrally by continuous infusion intracerebroventricularly (i.c.v.) using guide cannulas implanted i.c.v. connected to pumps implanted subcutaneously. The pumps deliver drug at a constant flow rate per day, however the animals gain weight throughout the course of the experiment. The doses reported are on a mg/kg/day basis and are based on the starting weights of the rats, and the concentration of drug and flow rates of the pumps. The corresponding drug exposure measurements were taken near the start of the experiment and thus correspond most closely to the indicated starting doses. The actual doses, on a mg/kg/day basis, are therefore approximately 25 to 30% lower by the end of the experiments. The brain to plasma ratio was much higher with i.c.v. administration, reaching a ratio of 6 at 0.00625 mg/kg/day and estimated to be greater than 9 at 0.002 mg/kg/day. The brain levels were 12 ng/g at 0.00625 mg/kg/day compared with 2 ng/ml (equal to 2 ng/g) in plasma. Significantly, 0.00625 mg/kg/day administered C.I. i.c.v. was effective in reversing the neuronal and behavioral deficits following 6-hydroxydopamine (6OHDA) lesions of the substantia nigra pars compacta (SNc) (see below). Similarly, a dose of 0.25 mg/kg/day administered C.I. s.c. resulted in brain bexarotene levels of 14 ng/g, suggesting that 0.25 mg/kg/day administered C.I. s.c. would also be an effective dose in reversing the neuronal and behavioral deficits following 6-hydroxydopamine (6OHDA) lesions of the substantia nigra pars compacta (SNc). However in this case the plasma levels of bexarotene were 12 ng/g; resulting in a brain/plasma ratio of 1.2. Finally a series of doses of bexarotene ranging from 1 to 100 mg/kg/day were administered as once daily oral doses (P.O. QD). Brain and plasma levels of bexarotene increased with dose in a dose-proportional manner from 1 to 10 mg/kg/day and in a slightly less than dose-proportional manner at 30 and 100 mg/kg/day. The brain/plasma ratio was consistently below 1, ranging between 0.4 and 0.8 at all doses tested.

Table 4 summarizes the dose/exposure/effect relationships for bexarotene in rodent models of Parkinson's disease and cancer compared to data from the Targretin NDA #21055. In addition, 60 mg/kg/day oral administration of bexarotene was effective in preventing tumor growth in nude mice injected with the cancer cell lines HN9N and HN21P (NDA #21055). These data show bexarotene is readily absorbed into the brain through various routes of administration and they define the minimum dose, exposure (AUC) and brain concentrations of bexarotene needed for efficacy in rat models of PD. In addition they demonstrate that substantially lower doses and exposure are required for efficacy in rodent models of PD than cancer. The plasma-brain profiles over time after oral dosing are shown in FIG. 5. At 1 mg/kg, brain concentrations of bexarotene remain higher than the minimum brain concentrations needed to reverse the neuronal and behavioral deficits as determined from i.c.v. and s.c. infusion experiments (see below). Also of note, the exposure was lower in lesioned rats than unlesioned rats (Table 4).

TABLE 4

| | | | | | | | Effective | Effective |
|---|---|---|---|---|---|---|---|---|
| | | Plasma | Brain | plasma | Brain | | in | in |
| Dosing | Dose | conc | conc | AUC | AUC | Brain/plasma | rat PD | rat cancer |
| route | (mg/kg/day) | (ng/ml) | (ng/ml) | (μM * hr) | (μM * hr) | ratio | model? | model? |
| i.c.v. | 0.006 | <2 | 12 | <0.1 | 0.8 | >6 | Yes | — |
| s.c. | 0.06 | 4 | — | 0.2 | — | — | No | — |
| s.c. | 0.25 | 12 | 14 | 0.8 | 1.0 | 1.2 | Yes | — |
| s.c. | 1 | 35 | 47 | 2.4 | 3.3 | 1.4 | Yes | — |
| p.o. | \hat{ }1 | 208 | 140 | 2.3 | 1.9 | 0.8 | Yes | — |
| p.o. | 1 | 220 | 110 | 3.0 | 1.9 | 0.6 | Yes | — |
| p.o. | *3 | 249 | — | 4.4 | — | — | — | — |
| p.o. | 10 | 1370 | 441 | 13.2 | 7.0 | 0.5 | — | — |
| p.o. | *10 | 541 | — | 14.1 | — | — | — | No |
| p.o. | *30 | 1162 | — | 24.3 | — | — | — | Yes/No |
| p.o. | *100 | 1888 | — | 42.1 | — | — | — | Yes |

*data from bexarotene NDA #21055.
AUC for s.c. dosing calculated using the trapezoidal rule.
AUC for p.o. dosing calculated using prizm software.
[—] denotes not measured.
The rat PD was 6OHDA lesioning of the substantia nigra and the cancer model was the NMU (N-nitroso-N-methylurea) induced mammary tumor carcinoma model (see NDA #21055).
Plasma and brain concentrations from i.c.v. and s.c. dosing are steady state levels after 4 to 8 days of continuous infusion. Plasma and brain concentrations from oral dosing experiments are peak concentrations obtained after 5 days of dosing, except data from NDA #21055 was after 15 to 50 days of dosing.
Yes/no indicates partial efficacy.
Brain/plasma ratio = AUC brain/AUC plasma.
\hat{ }PK performed in 6OHDA lesioned rats.

### Example 4

### Bexarotene Efficacy when Administered i.c.v.

This example illustrates evaluation of bexarotene efficacy when administered i.c.v. after 6-OHDA lesion to assess neuroregenerative potential of bexarotene. The endpoints assessed were:
Neuroprotection measured by tyrosine hydroxylase (TH) staining in the substantia nigra pars compacta (SNc) and dopamine transporter (DAT) and vesicular monoamine transporter 2 (VMAT2) staining in the striatum (STR).
Behavioral assessments including rotorod, challenging beam, and spontaneous locomotion

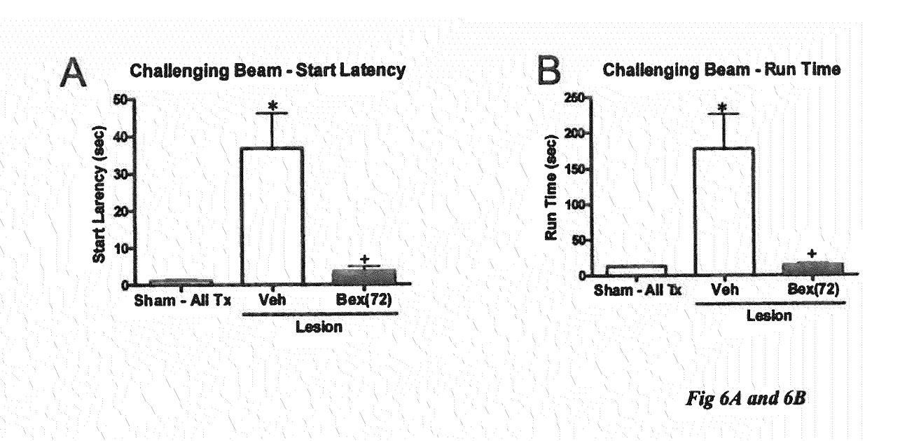

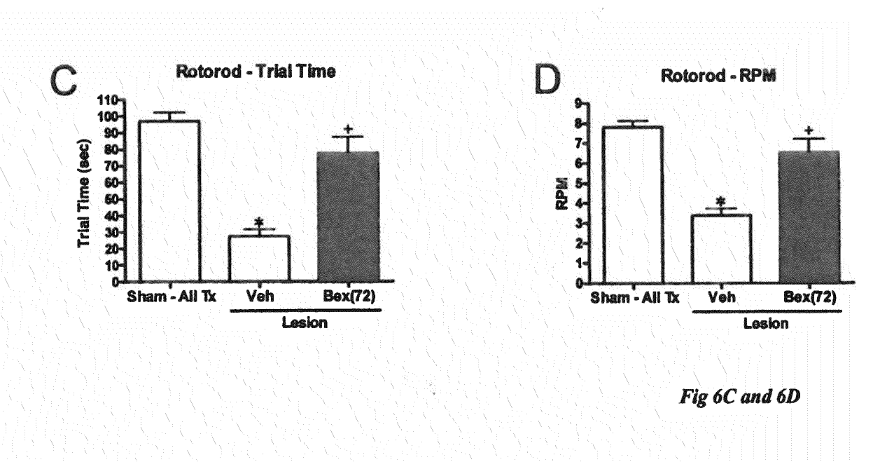

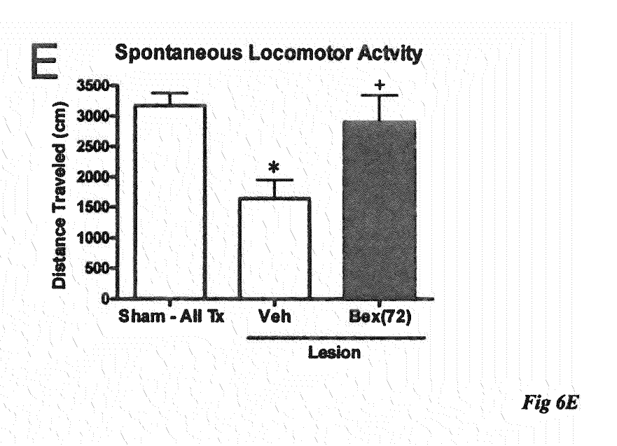

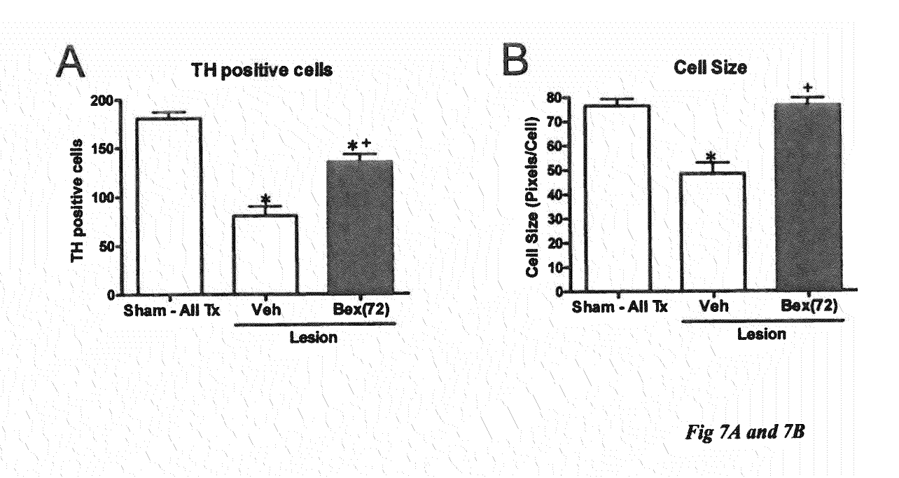

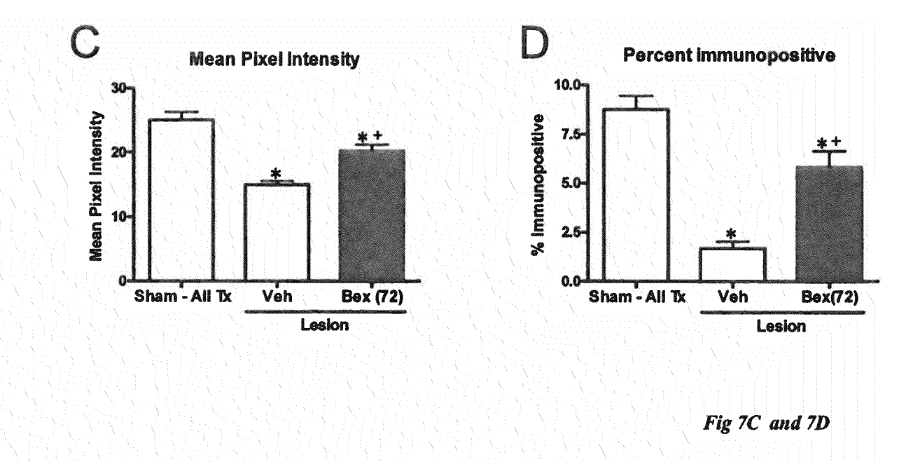

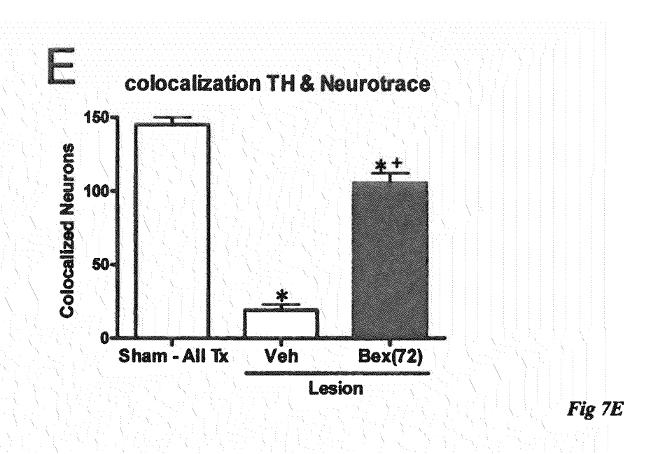

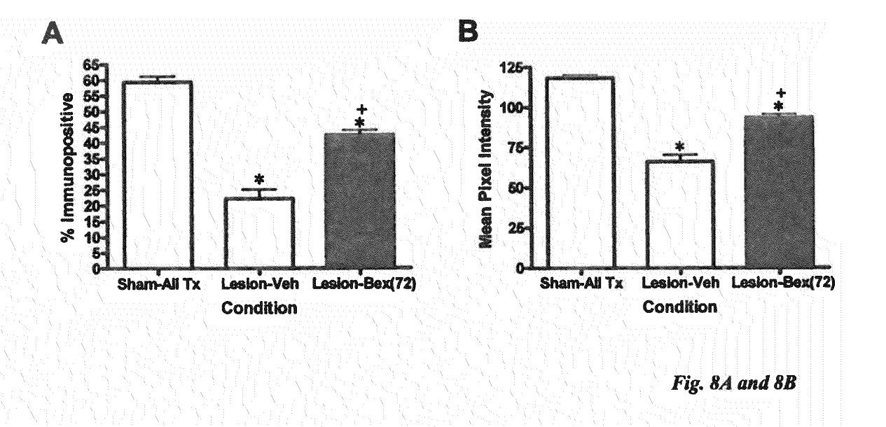

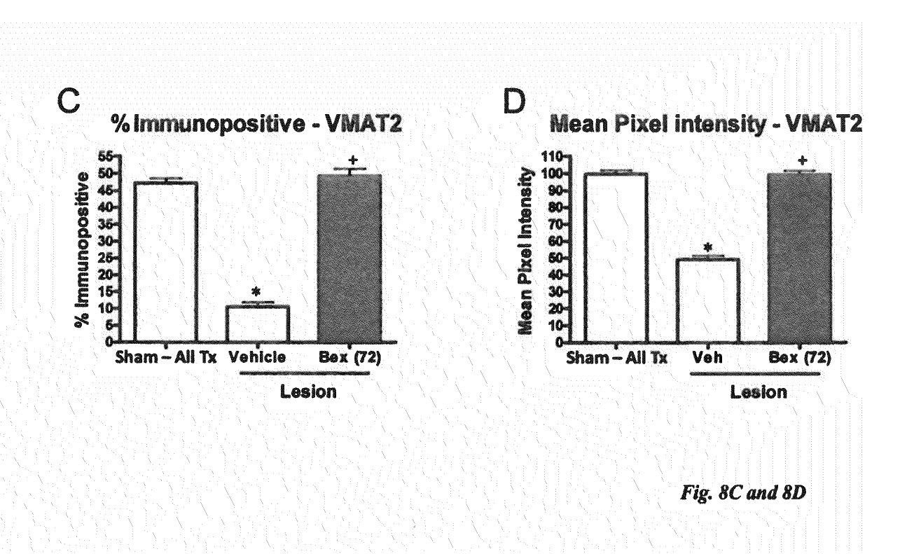

Bexarotene was tested for its ability to slow down, stop or even reverse neuronal and behavioral deficits following 6-hydroxydopamine (6OHDA) lesions of the substantia nigra pars compacta (SNc). 6OHDA was infused bilaterally into the SNc of male rats to produce destruction of dopamine neurons. Using an osmotic pump, bexarotene or vehicle was infused into the cerebral ventricle at a constant rate (0.25 μL/hr or 6 μL/day of a 1 mM solution of bexarotene providing a dose of 0.000625 mg/kg/day, see Table 4 above) for 28 days beginning 72 hours after 6OHDA infusion. Following the 28 days of treatment, animals were assessed in 3 tests of coordinated motor function (spontaneous locomotion, rotorod and challenging beam) and then tissue was collected to assess tyrosine hydroxylase immunofluorescence in the substantia nigra (SNc) and DAT and VMAT2 in the striatum (STR). Treatment with bexarotene reversed behavioral deficits caused by 6OHDA administration (see FIG. 6), and resulted in improved tyrosine hydroxylase expression in the SNc (see FIG. 7), improved dopamine transporter and VMAT2 expression in the STR (see FIG. 8).

This example indicates that bexarotene displays efficacy in both neuroregeneration and behavioral endpoints when administered after 6-OHDA lesioning.

### Methods

Subjects:

The subjects for these experiments were male Sprague-Dawley rats purchased from Charles Rivers Laboratories (Hollister, Calif.) weighing 200-225 g upon arrival. Rats were housed in pairs in polypropylene cages within a temperature controlled vivarium maintained on a 12 hr light:dark cycle (lights on 7 am). For the duration of the experiments, animals received free access to food and water. All procedures were conducted in accordance with the NIH Guidelines for the Care and Use of Laboratory Animals and were approved by the Institutional Animal Care and Use Committee (IACUC) at ACADIA Pharmaceuticals. Animals were acclimated to vivarium conditions and handling for a minimum of one week prior to surgery.

Surgery:

In order to protect norepinephrine terminals, each animal received an injection of desipramine (10 mg/kg) about 15 min prior to being anesthetized using isofluorane. Animals were placed into a stereotaxic apparatus and bilateral infusions of 6OHDA (8 μg/4 μl) or 0.2% ascorbic acid vehicle were aimed at the SNc (A/P −5.2 mm, M/L±1.6 mm, D/V −8.0 mm relative to bregma). After 6OHDA infusions, an Alzet osmotic pump (Durect Corporation, Cupertino, Calif.) attached to an intracranial guide cannula was implanted subcutaneously between the shoulder blades of each animal. The guide was placed intracerebroventricularly (i.c.v., A/P −0.8 mm, M/L −1.4 mm, D/V −4.5 mm relative to bregma) and was attached to the skull with jeweler's screws and dental acrylic and the incision was closed with staples. Animals received supportive care following surgery, including administration of subcutaneous (sc) fluids (10 ml/day) and soft food mashes, until they surpassed their surgical weights. Subjects were allowed at least 28 days prior to behavioral testing:

Pumps:

The osmotic pumps (Alzet, model 2004) were weighed and then filled with bexarotene (1 mM) or vehicle (1% DMSO in saline) 48 hours prior to surgery. They were then incubated in 0.9% physiological saline at 37° C. until surgically implanted. The pumps infused at a rate of 0.25 μL/hr for 28 days after implantation. Infusion pumps were connected to the i.c.v. cannula with vinyl tubing and different infusion conditions were achieved by filling the tubing with varying amounts of vehicle before bexarotene reached the guide. Thus, bexarotene infusion began 72 hours after implantation of the guide and the following surgery/treatment conditions were employed (N=3-5/group): Sham/vehicle, Sham/bexarotene (72), 6OHDA/vehicle, 6OHDA/bexarotene (72). After completion of the experiment, a subset of the osmotic pumps was removed. The pumps were weighed and aspirated in order to verify compound delivery. For all pumps tested, this procedure confirmed that the pumps successfully delivered compound.

Spontaneous Locomotion:

Locomotor activity studies were conducted in acrylic chambers (42 cm×42 cm×30 cm) equipped with 16 infrared photobeams along each horizontal axis (front-to-back and side-to-side) from Accuscan Instruments, Inc. (Columbus, Ohio). Animals were placed into the chamber for 15 min and their distance traveled (cm) was recorded.

Rotorod:

Rotorod testing was conducted on a rotating cylinder (70 mm diameter) with knurled tread to aid in gripping. Animals were placed on the cylinder and it was set to rotate at 1 rpm for 15 sec. If animals fell or jumped from the cylinder within 30 sec., they were replaced and the acclimation period restarted. Once animals successfully remained on the cylinder for the acclimation period, the speed of rotation was increased 1 rpm every 15 sec to a maximum of 10 rpm. The time in seconds that animals remained on the cylinder after the acclimation period and the maximum rpm achieved were recorded. A second trial was conducted after a 2 min intertrial interval using the same procedure, but the acclimation period was decreased such that animals were only required to step with all four feet before the speed of rotation was increased. Data are from Trial 2.

Challenging Beam Test:

The challenging beam test was conducted on a 102 cm long bi-level beam made from ABS plastic. The top, narrower beam gradually tapered from 3.5 cm to 0.7 cm, while the bottom, wider beam gradually tapered from 5 cm to 1.8 cm along the length of the beam. The beam was elevated 23 cm above the table. Animals were placed in groups of 4 into a holding tub and received five training trials. On the first training trial animals were placed at the end of the beam and were required to jump into a holding tub. On successive trials, animals were placed 25, 50, 75 and 100 cm from the end of the beam and were required to traverse the beam and jump into the holding tub at the end. Following training, a single test trial was conducted where each animal was placed at the beginning of the beam and the start latency (time required to move all four feet from their starting locations) and run time (time required to traverse the beam after starting) were recorded. Animals were allowed a maximum of 300 seconds to traverse the beam, at which point they were removed from the beam and a run time of 300 sec was recorded.

Tyrosine Hydroxylase Fluorescent Immunohistochemisty: Following behavioral testing, animals were anesthetized and perfused transcardially with PBS followed by 4% paraformaldehyde. Fixed tissue brains were sectioned (50 μm) through the subtantia nigra and then were immunolabeled for tyrosine hydroxylase using the following steps: 3×5 min rinses in 1× phosphate buffered saline (PBS); 45 min blocking step in blocking buffer (0.8 PBS, 3% normal donkey serum, 0.1% Triton); incubation with rabbit anti-tyrosine hydroxylase polyclonal antibody (AB152, Millipore Corp., Billerica, Mass.) in working buffer (1×PBS, 1% blocking buffer, 0.1% Triton) for 2 hr at room temperature; 3×5 min rinses in working buffer; incubation with donkey anti-rabbit Alexa Fluor 488 fluorescent secondary antibody (A21206, Invitrogen Corp., Carlsbad, Calif.) in working buffer for 1 hr; 3×5 min rinses in working buffer.

Dopamine Transporter Immunohistochemistry:

Similarly, fixed brains were sectioned (50 μm) through the striatum and labeled for the dopamine transporter. The dopamine transporter was labeled with DAB immunohistochemistry using the following steps: 3×5 min rinses in 1× phosphate buffered saline (PBS); 20 min incubation in sodium citrate buffer (10 mM sodium citrate in 1×PBS, 0.05% Tween 20, pH=6.0) at 80 C to promote antigen retrieval; 10 min incubation in 3% hydrogen peroxide to block peroxidase binding sites; 1 hour protein blocking step in blocking buffer (1×PBS, 8% normal goat serum, 3% bovine serum albumin, 0.1% Triton, avidin blocking solution from Vector Laboratories, Burlingame, Calif.); incubation with rat anti-dopamine transporter monoclonal antibody (MAB369, Millipore Corp.) in a working buffer (1×PBS, 2% normal goat serum, 1% bovine serum albumin, biotin blocking solution from Vector Laboratories) overnight at 4 C; 3×5 min rinses in 1×PBS; incubation with goat anti-rabbit biotinylated secondary antibody (BA-9400, Vector Laboratories) in working buffer without biotin for 1 hr; 3×5 min rinses in 1×PBS; 30 min incubation in ABC wash (PK-6100, Vectastain Elite ABC kit, Vector Laboratories); 3×5 min rinses in 1×PBS; 5 min DAB (3,3′-diaminobenzidine) incubation (SK-4100, DAB substrate kit, Vector Laboratories); 1×5 min rinse in ddH₂O; 2×5 min rinses in 1×PBS (Phosphate buffered saline). The sections were then mounted on slides and allowed to dry before being submerged in successive 3 min washes (70% EtOH, 95% EtOH, 100% EtOH, 50/50 Citrisolve/EtOH, 100% Citrisolve) and coverslipped using a xylene-based permanent mounting medium (H-5000, VectaMount, Vector Laboratories).

Vesicular Monoamine Transporter 2 Immunohistochemistry:

VMAT2 was labeled with DAB immunohistochemistry using the following steps: 3×5 min rinses in 1× phosphate buffered saline (PBS); 10 min incubation in 3% hydrogen peroxide to block peroxidase binding sites; 3×5 min rinses in 1× phosphate buffered saline (PBS); 1 hour protein blocking step in blocking buffer (1×PBS, 8% normal goat serum, 3% bovine serum albumin, 0.25% Triton, avidin blocking solution from Vector Laboratories, Burlingame, Calif.); incubation with rabbit anti-VMAT2 polyclonal antibody (NB100-68123, Novus Biologicals) in a working buffer (1×PBS, 2% normal goat serum, 1% bovine serum albumin, 0.2% Triton, biotin blocking solution from Vector Laboratories) overnight at 4 C; 3×5 min rinses in 1×PBS; incubation with goat anti-rabbit biotinylated secondary antibody (BA-1000, Vector Laboratories) in working buffer without biotin for 1 hr; 3×5 min rinses in 1×PBS; 30 min incubation in ABC wash (PK-6100, Vectastain Elite ABC kit, Vector Laboratories); 3×5 min rinses in 1×PBS; 5 min DAB incubation (SK-4100, DAB substrate kit, Vector Laboratories); 1×5 min rinse in ddH2O; 2×5 min rinses in 1×PBS.

After immuno labeling sections were mounted and coverslipped using fluorescent antifade mounting medium (S3023, Dako USA, Carpinteria, Calif.). Single optical plane images were obtained using an Olympus BX51 Fluorescent microscope (Olympus America Inc., Center Valley, Pa.) equipped with a digital camera (Retina 2000R, Qimaging, Surrey, BC). Images were acquired using a 4× air objective (UPlanFL N, N.A. 0.13) with 2× digital magnification. For each animal 3 consecutive sections through each SNc were analyzed (−5.2 mm relative to bregma according to the atlas of Paxinos and Watson, 1997). All images (N=6/animal) were treated as independent observations and analyzed by an observer blind to each subject's treatment condition using ImageJ software (available at http://rsb.info.nih.gov/nih-imageJ, developed by Wayne Rasband at NIH, Bethesda, Md.) in order to determine the cell count (SNc tissue only), cell size (pixels/cell, SNc tissue only), pixel intensity, and % immunopositive. Data represent the mean SNc section for these measures across different treatment conditions. Controls were performed via omission of the primary antibody and revealed no non-specific staining (data not shown).

Confirmation of drug delivery. Animals receiving bexarotene i.c.v. by osmotic pumps were sacrificed at 3 weeks, brains harvested, and analyzed for bexarotene using LC-MS/MS according to the vendor's (Agilux Laboratories) procedures. The results of this study are shown in Table 4 above.

### Example 5

### Bexarotene Efficacy when Administered Systemically Through the Subcutaneous Route

This example illustrates evaluation of bexarotene efficacy when administered s.c. after 6-OHDA leasion to assess neuroregenerative potential of bexarotene. The endpoints assessed were:
Neuroprotection measured by tyrosine hydroxylase (TH) and ret-c (co-receptor for the trophic factor GDNF) staining in the substantia nigra (SNc).
Behavioral assessments including rotorod, challenging beam, and spontaneous locomotion

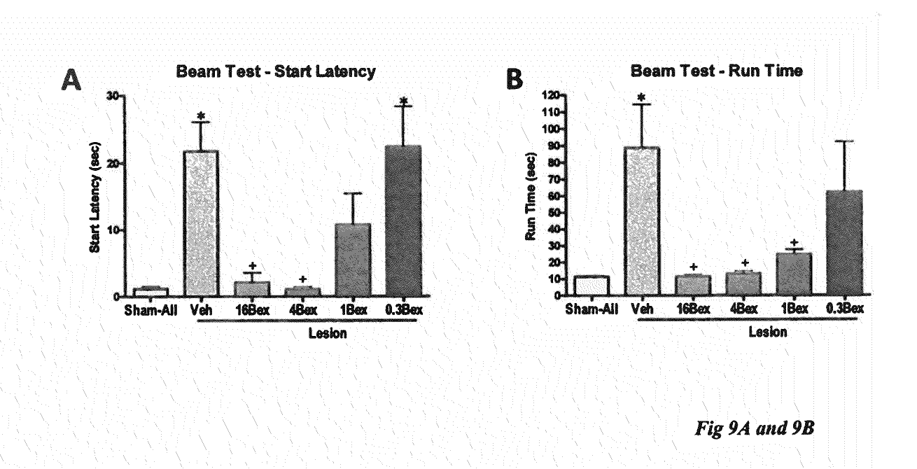

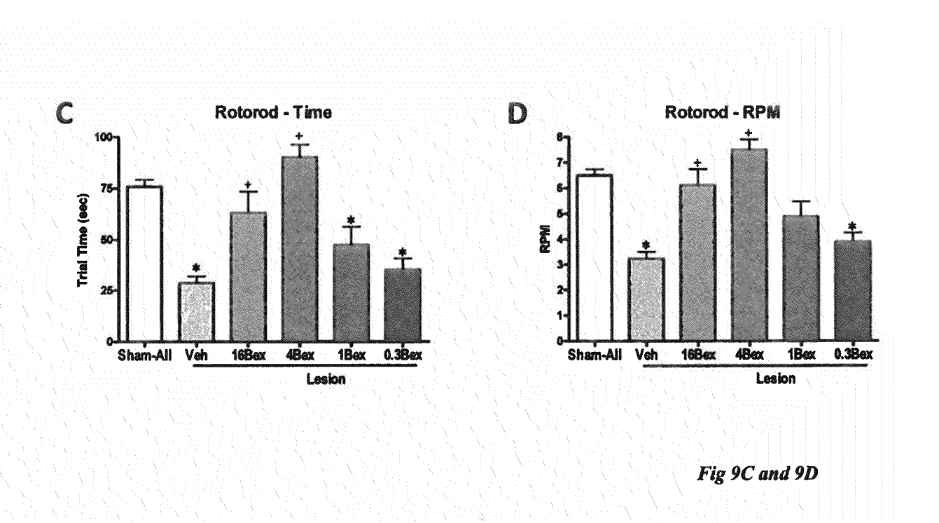

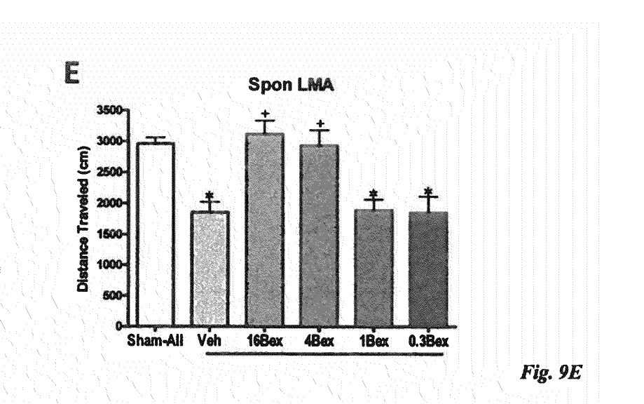

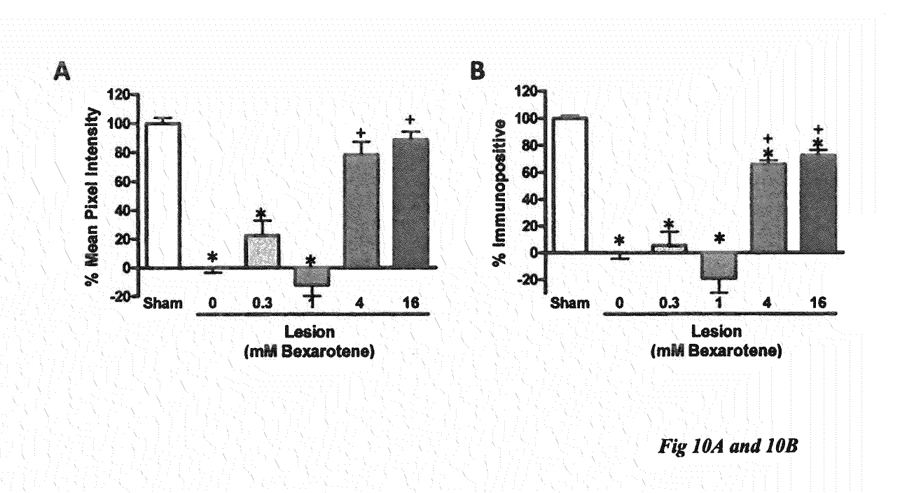

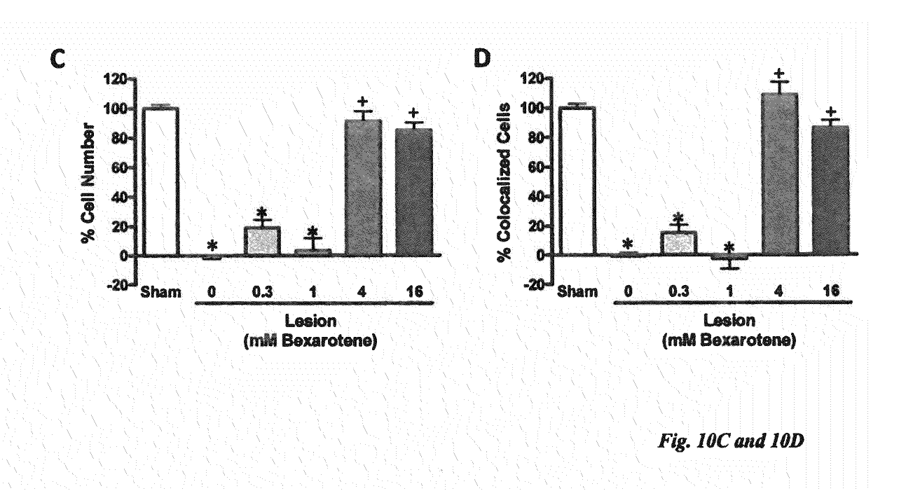

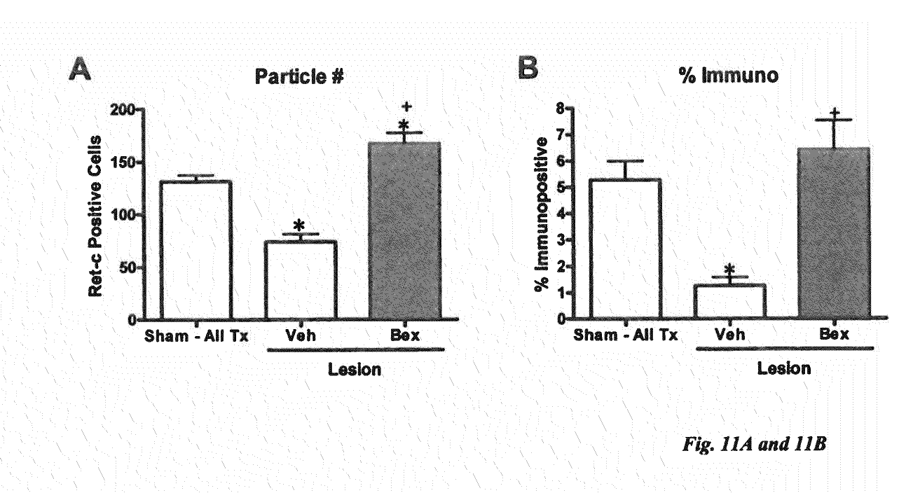

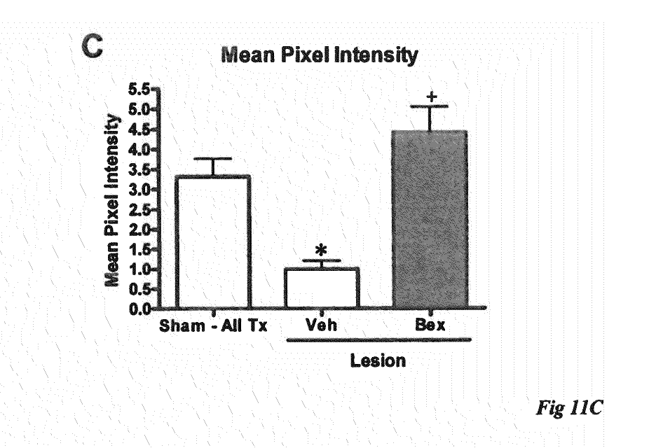

Bexarotene was tested for its ability to reverse neuronal and behavioral deficits following 6-hydroxydopamine (6OHDA) lesions of the substantia nigra pars compacta (SNc). 6OHDA was infused bilaterally into the SNc of male rats to produce destruction of dopamine neurons. Using an osmotic pump implanted on the dorsal side between the scapulae, bexarotene or vehicle was infused subcutaneously at a constant rate (2.5 μL/hr or 60 μL/day of a 16, 4, 1, or 0.3 mM solution of bexarotene providing a dose of 1, 0.25, 0.0625 or 0.021 mg/kg/day, see Table 4 above) for 28 days beginning 72 hours after 6OHDA infusion. Following the 28 days of treatment, animals were assessed in 3 tests of coordinated motor function (spontaneous locomotion, rotorod and challenging beam) and then tissue was collected to assess tyrosine hydroxylase and Ret-c immunofluorescence in the substantia nigra. Treatment with bexarotene reversed behavioral deficits caused by 6OHDA administration (see FIG. 9), and resulted in improved tyrosine hydroxylase and Ret-c expression in the SNc (see FIGS. 10 and 11).

This example indicates that bexarotene displays efficacy in both neuroregeneration and behavioral endpoints when administered systemically though the continuous infusion subcutaneously after 6-OHDA lesioning.

### Methods

The Subjects and Surgical Procedures to Produce Lesions were as Described Above for i.c.v. Dosing.

Pumps:

The osmotic pumps (Alzet, model 2ML4) were weighed and then filled with bexarotene (16, 4, 1 or 0.3 mM) or vehicle (50% DMSO:50% PEG400) 48 hours prior to surgery. They were then incubated in 0.9% physiological saline at 37° C. until surgically implanted. The pumps infused at a rate of 2.5 μL/hr for 28 days after implantation. The 16, 4, 1, or 0.3 mM solutions of bexarotene provided doses of 1, 0.25, 0.0625 or 0.021 mg/kg/day. Infusion pumps were connected to the s.c. cannula with vinyl tubing and different infusion conditions were achieved by filling the tubing with varying amounts of vehicle before bexarotene reached the guide. Thus, bexarotene infusion began 72 hours after implantation of the guide and the following surgery/treatment conditions were employed (N=10/group): Sham/vehicle, Sham/bexarotene(16), 6OHDA/vehicle, 6OHDA/bexarotene(16), 6OHDA/bexarotene (4), 6OHDA/bexarotene (1), and 6OHDA/bexarotene (0.3). After completion of the experiment, a subset of the osmotic pumps was removed. The pumps were weighed and aspirated in order to verify compound delivery. For all pumps tested, this procedure confirmed that the pumps successfully delivered compound.

Spontaneous Locomotion, rotorod, and challenging beam tests were conducted as described above for i.c.v. dosing.

Tyrosine Hydroxylase Fluorescent Immunohistochemistry was conducted as described above for i.c.v. dosing. Ret-c immunohistochemistry was conducted using brains fixed and SNc tissue sectioned as described above. Ret-c was labeled with DAB immunohistochemistry using the following steps: 3×5 min rinses in 1× phosphate buffered saline (PBS); 10 min incubation in 3% hydrogen peroxide to block peroxidase binding sites; 3×5 min rinses in 1×PBS; 20 min incubation in sodium citrate buffer (10 mM sodium citrate in 1×PBS, 0.05% Tween 20, pH=6.0) at 80 C to promote antigen retrieval; 3×5 min rinses in 1×PBS; 2 hour protein blocking step in blocking buffer (1×PBS, 8% normal goat serum, 3% bovine serum albumin, 0.1% Triton, avidin blocking solution from Vector Laboratories, Burlingame, Calif.); incubation with rabbit anti-ret polyclonal antibody (Santa Cruz, sc-167) in a working buffer (1×PBS, 2% normal goat serum, 1% bovine serum albumin, biotin blocking solution from Vector Laboratories) overnight at 4 C; 3×5 min rinses in 1×PBS; incubation with goat anti-rabbit biotinylated secondary antibody (BA-1000, Vector Laboratories) in working buffer without biotin for 1 hr at RT; 3×5 min rinses in 1×PBS; 30 min incubation in ABC wash (PK-6100, Vectastain Elite ABC kit, Vector Laboratories); 3×5 min rinses in 1×PBS; 5 min DAB incubation (SK-4100, DAB substrate kit, Vector Laboratories); 1×5 min rinse in ddH2O; 2×5 min rinses in 1×PBS. Ret-c sections were then mounted on and imaged as described above for immunohistochemistry performed on TH.

### Example 6

### Bexarotene Efficacy when Administered Orally

This example illustrates evaluation of bexarotene efficacy when administered once per day orally after 6-OHDA lesion to assess neuroregenerative potential of bexarotene. The endpoints assessed were:
Neuroprotection measured by tyrosine hydroxylase (TH) staining in the substantia nigra (SNc).
Behavioral assessments including rotorod, challenging beam, and spontaneous locomotion

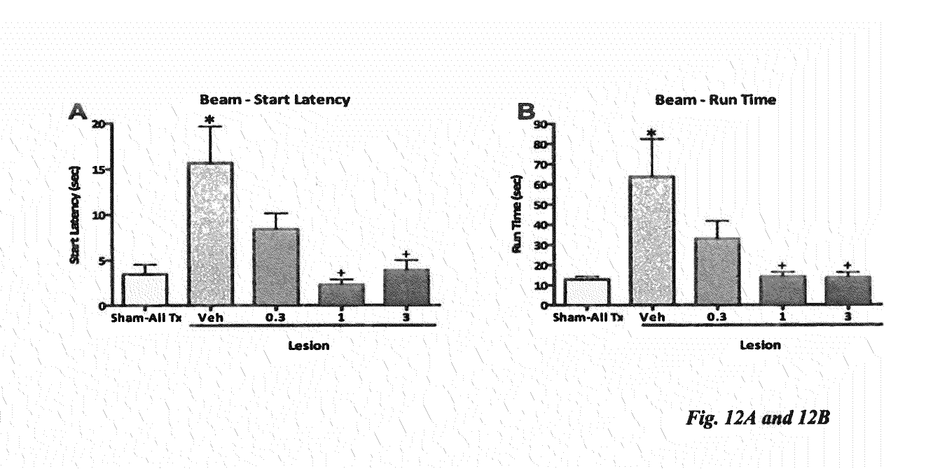

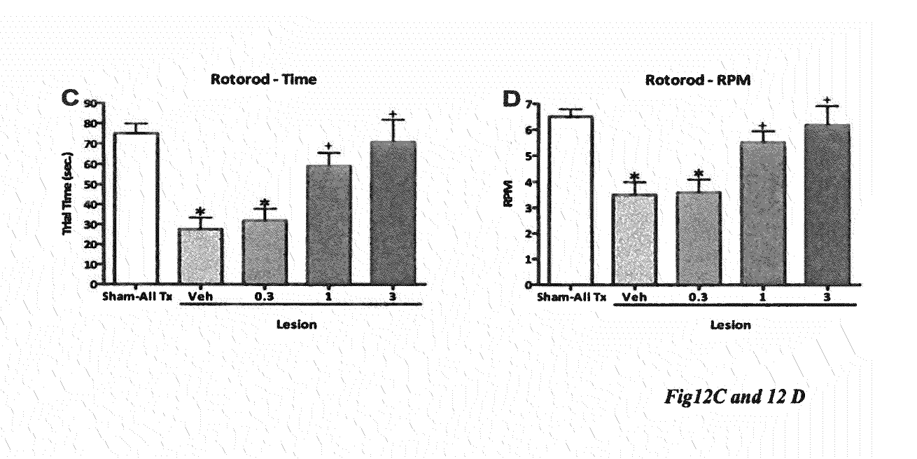

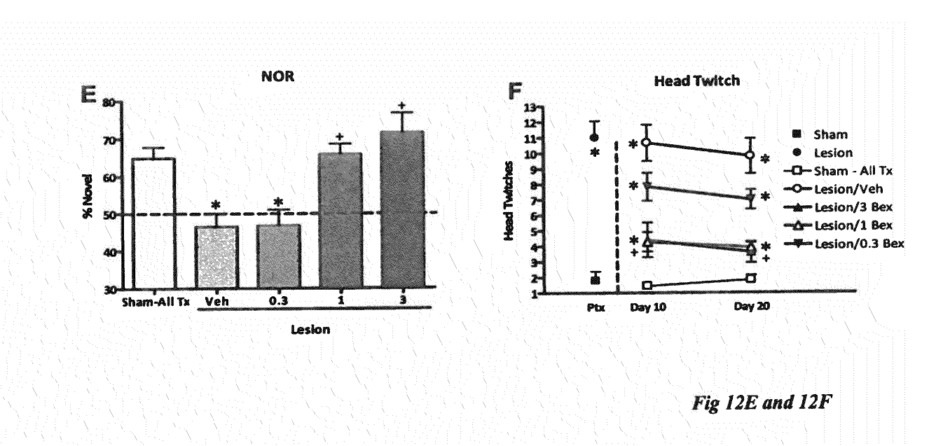

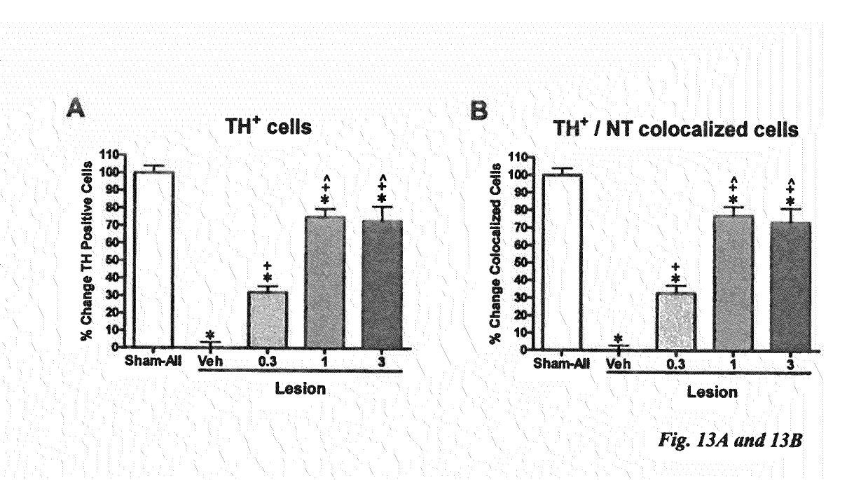

Bexarotene was tested for its ability to reverse neuronal and behavioral deficits following 6-hydroxydopamine (6OHDA) lesions of the substantia nigra pars compacta (SNc). 6OHDA was infused bilaterally into the SNc of male rats to produce destruction of dopamine neurons. Bexarotene (1 or 3 mg/kg/day) or vehicle was administered once per day orally see Table 4 and FIG. 5 above) for 28 days beginning 72 hours after 6OHDA infusion. Following the 28 days of treatment, animals were assessed in 3 tests of coordinated motor function (spontaneous locomotion, rotorod and challenging beam) and then tissue was collected to assess tyrosine hydroxylase immunofluorescence in the substantia nigra. Treatment with bexarotene reversed behavioral deficits caused by 6OHDA administration (see FIG. 12), and resulted in improved tyrosine hydroxylase expression in the SNc (see FIG. 13).

This example indicates that bexarotene displays efficacy in both neuroregeneration and behavioral endpoints when administered orally after 6-OHDA lesioning.

### Methods

The subjects and surgical procedures to produce lesions were as described above for i.c.v. dosing.

Spontaneous Locomotion, rotorod, and challenging beam tests were conducted as described above for i.c.v. dosing.

Novel object recognition (NOR) was conducted in a novel environment in two phases: sample and test. Subjects were placed into the NOR chamber, where two identical objects were placed. Each rat was allowed to explore for 3 min., and the time spent exploring at each position recorded. After 3 min., each rat was removed from the arena and placed back into its cage. The test phase was conducted 4 hours after the sample phase. During test, one familiar object (seen during sample) and one novel object was placed into the chamber, and each rat was allowed 3 min to explore. The test sessions were recorded on video and scored by an observer blind to each subject's treatment condition. For test data, % of exploration time spent at the novel object was determined and headtwitch assays.

Spontaneous Head Twitch: Subjects were place in a group of 4 animals into a clean holding tub, where they were closely observed for 8 min. A head twitch was counted each time an animal displayed a rapid, bidirectional head movement or “wet dog shake” that was unrelated to grooming or exploration.

Tyrosine Hydroxylase Fluorescent Immunohistochemisty was conducted as described above for i.c.v. dosing.

### Example 7

### Bexarotene Regenerates Neurons

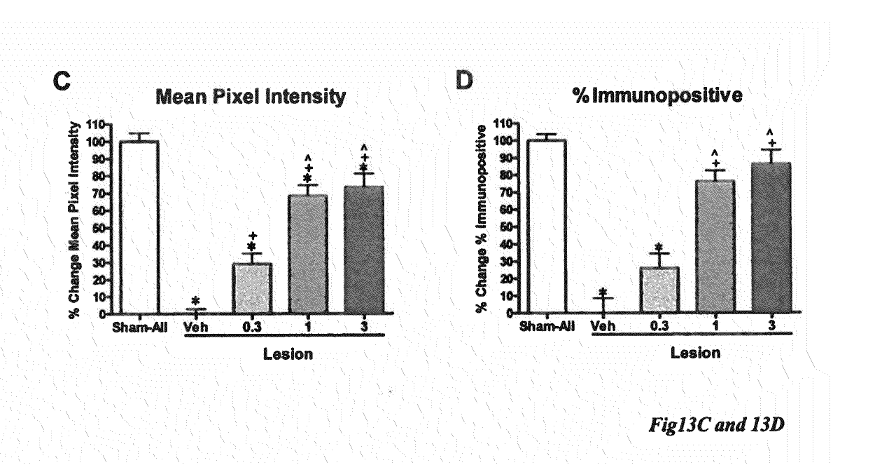

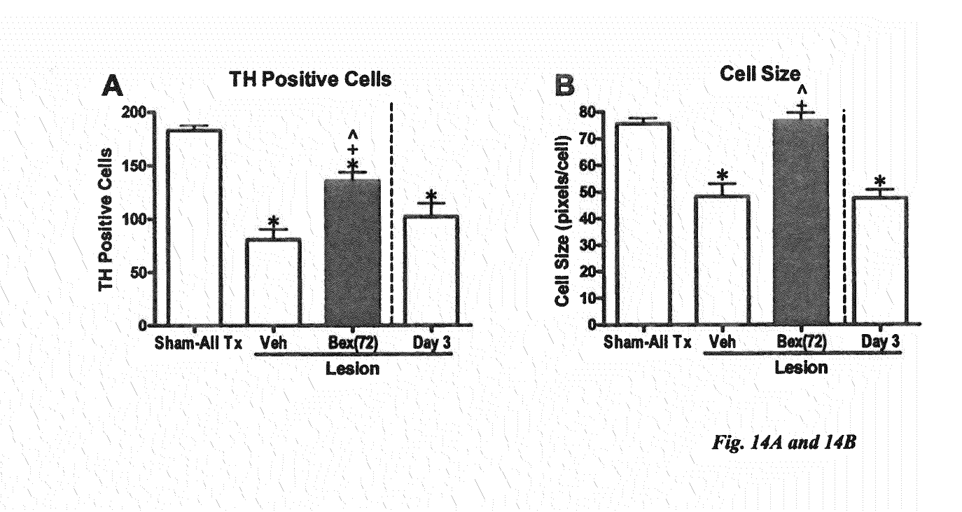

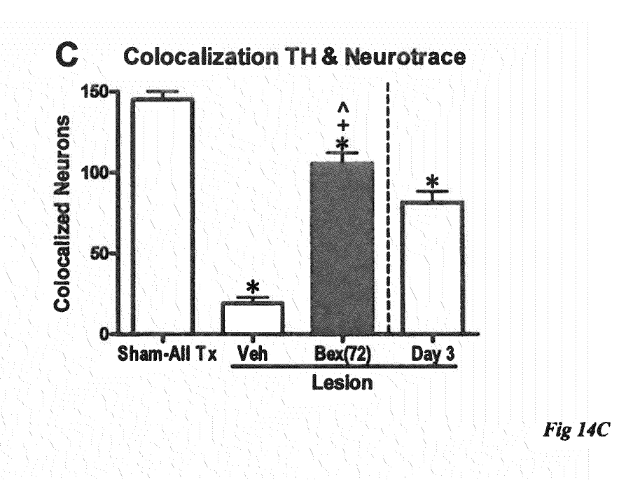

In this example (see FIG. 14) animals received vehicle (Sham-All Tx) or 6OHDA treatment (Lesion) bilaterally into the SNc. 3 days after surgery, animals were either sacrificed (Day 3) or began receiving bexarotene (1 mM, 0.25 μL/hr) or vehicle (1% DMSO) intracerebroventricularly for 28 days. Compared with sham controls, 6OHDA treated animals (Lesion/Veh) displayed a reduced number of TH positive cells in the SNc (Panel A), reduced mean cell size (Panel B), and a reduced colocalization of TH with the neuronal marker Neurotrace. Treatment with bexarotene beginning 72 hours after 6OHDA lesion (Lesion/Bex(72)) significantly improved the number of TH positive cells, cell size and colocalization of TH and Neurotrace. Notably, bexarotene treatment also significantly improved all of these measures when compared with animals sacrificed 3 days after lesion (i.e. at the start of bexarotene treatment). Data were analyzed with one-way ANOVAs followed by post hoc Tukey's multiple comparisons test. * indicates a significant difference from Sham, p<0.05; + indicates a significant difference from vehicle/6OHDA, p<0.05; ´ indicates a significant difference from Day 3, p<0.05.

The results are shown in FIG. 14.

### Example 8

### Effect of Bexarotene after a MPP+ Injury in Rat Primary Dopaminergic Neurons

The neurotoxicant 1-methyl-4-phenyl-1,2,3,6-tetrahydropyridine (MPTP) is a specific dopaminergic neuronal toxin. MPTP is converted to 1-methyl-4-phenyl pyridinium (MPP+) by astroglia and then causes specific dopaminergic neuronal death in the SNc, thus leading to the clinical symptoms of PD in humans, primates and mice (Uhl et al., 1985). For this reason, MPTP-induced dopaminergic neurotoxicity in mice is widely used as a model for PD research. It has been largely reported that MPP+ causes neurodegeneration of dopaminergic neurones in vitro and provides a useful model of Parkinson's disease in vitro.

The neurotrophins brain derived neurotrophic factor (BDNF) and glial derived neurotrophic factor (GDNF) have been suggested to reduce the MPP+− induced neurodegeneration in vitro (Hung & Lee, 1996); (Hou et al., 1996).

This example investigated the restorative effect of bexarotene tested at 7 concentrations on rat primary mesencephalic cultures previously injured by a 24 h exposure to 1-methyl-4-phenylpyridinium (MPP+), a Parkinson' disease model in vitro. BDNF was used as a positive control in this study.

### Experimental Protocol

### Primary Cultures of Dopaminergic Neurons

Rat dopaminergic neurons were cultured as described by Schinelli et al., 1988. Briefly pregnant female rats of 15 days gestation were killed by cervical dislocation (Rats Wistar; Janvier) and the foetuses removed from the uterus. The embryonic midbrains were removed and placed in ice-cold medium of Leibovitz (L15; PAN) containing 2% of Penicillin-Streptomycin (PS; Invitrogen) and 1% of bovine serum albumin (BSA; PAN). Only the ventral portions of the mesencephalic flexure were used for the cell preparations as this is the region of the developing brain rich in dopaminergic neurons. The midbrains were dissociated by trypsinisation for 20 min at 37° C. (Trypsin EDTA 1×; PAN) diluted in PBS without calcium and magnesium. The reaction was stopped by the addition of Dulbecco's modified Eagle's medium (DMEM; PAN) containing DNAase I grade II (0.5 mg/ml; PAN) and 10% of fetal calf serum (FCS; Gibco). Cells were then mechanically dissociated by 3 passages through a 10 ml pipette. Cells were then centrifuged at 180×g for 10 min at +4° C. on a layer of BSA (3.5%) in L15 medium. The supernatant was discarded and the cells of pellet were re-suspended in a defined culture medium consisting of Neurobasal (Gibco) supplemented with B27 (2%; Gibco), L-glutamine (0.2 mM; Invitrogen) 2% of PS solution and 10 ng/ml BDNF (PAN) and 1 ng/ml GDNF. (PAN) Viable cells were counted in a Neubauer cytometer using the trypan blue exclusion test. The cells were seeded at a density of 40000 cells/well in 96 well-plates (wells were pre-coated with poly-L-lysine (greiner) and were cultured at 37° C. in a humidified air (95%)/CO2 (5%) atmosphere. Half of the medium was changed every 2 days with fresh medium.

### MPP+ Exposure and Drug Treatment: Restorative Protocol

Briefly, on day 6 of culture, the medium was removed and fresh medium was added, without or with MPP+ at 4 μM. On day 7, the culture was washed with fresh medium without (containing vehicle) or with bexarotene (0.3, 1, 3, 10, 30, 100 and 300 nM) or BDNF (50 ng/ml) for 48 h. After 48 h with or without bexarotene (0.3, 1, 3, 10, 30, 100 and 300 nM) or BDNF (50 ng/ml), cells were fixed (all conditions) by paraformaldehyde 4% solution. In all wells, the final concentrations were in 0.1% DMSO. After permeabilization with 0.1% saponin (Sigma), cells were incubated with mouse monoclonal primary against tyrosine hydroxylase antibody (TH, Sigma) to stain specifically dopaminergic neurons. This antibody was revealed with Alexa Fluor 488 goat anti-mouse IgG (Molecular probe). The number of TH neurons and total neurite of TH neurons were measured in each well.

### Analysis and Method of Quantification

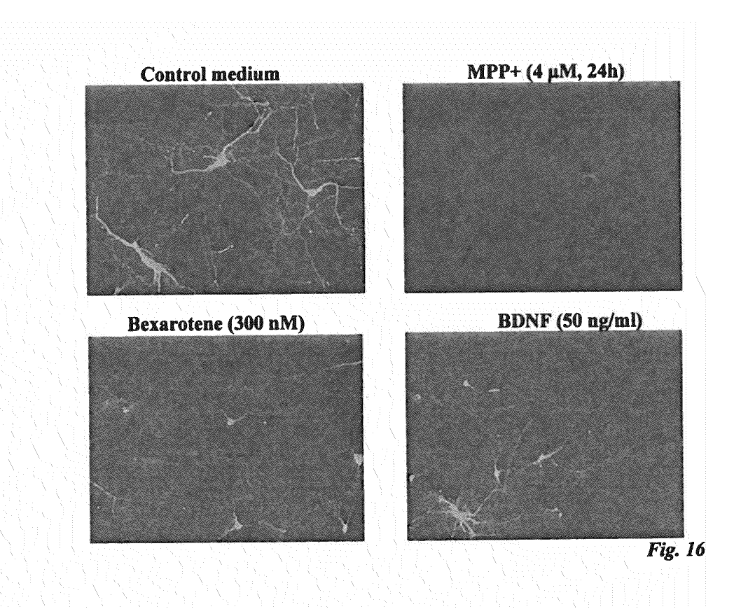

For each condition, 2×10 pictures per well were taken in the same condition using InCell Analyzer™ 1000 (GE Healthcare) with 10× magnification. The analyses were automatically done using developer software (GE Healthcare) to measure the total number of TH positive neurons. Two means of 10 pictures were automatically performed by well. Data were expressed in percentage of control condition. Statistical analyses (using Graph Pad Prism's package) were done on the different conditions using ANOVA test following by Dunnett's test (when allowed), significance was set for p≦0.05. Representative pictures are shown in FIG. 16.

In addition, the control/vehicle, MPP+/vehicle, MPP+/bexarotene (300 nM), and MPP+/BNDF (50 ng/ml) conditions were analyzed for number of neuron displaying neurites. For each condition, 10 pictures per well were taken in the same condition using InCell Analyzer™ 1000 (GE Healthcare) with 10× magnification. The analyses were done manually to measure the number of TH positive neurons displaying neurites. 6 wells per conditions were analyzed. Data were expressed in percentage of control condition. Statistical analyses (using Graph Pad Prism's package) were done on the different conditions using one-way ANOVA test following by Dunnett's test (when allowed: p<0.01), significance was set for p≦0.05.

### Results

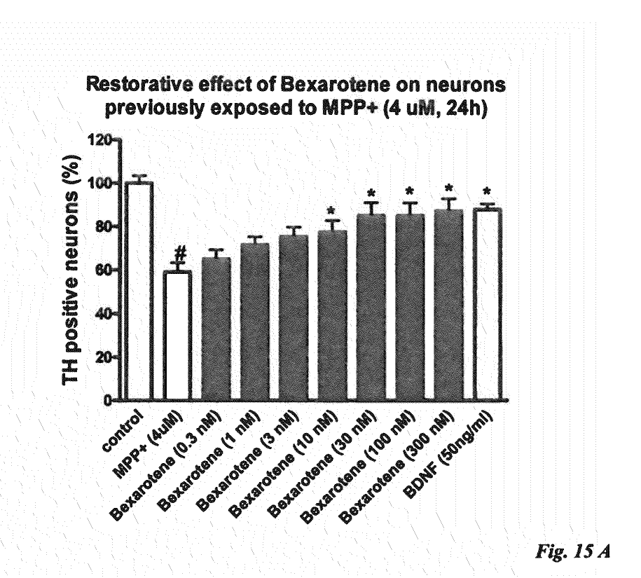

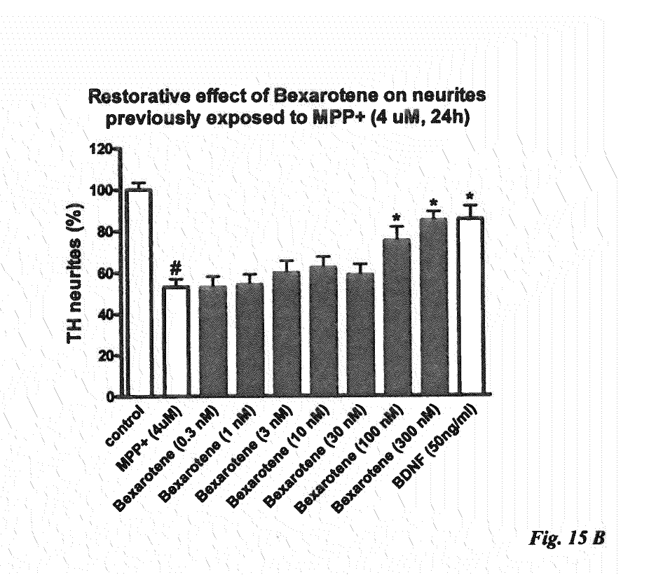

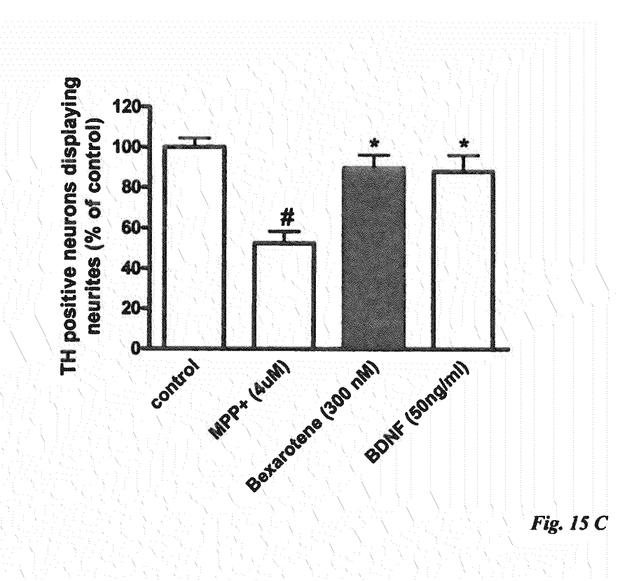

MPP+ at 4 μM (24 h intoxication) showed a large and significant TH positive neuron and TH positive neurite decreases. bexarotene applied after the 24 h intoxication displayed an increase of the total number of TH neurons at all tested concentrations. This restorative effect was significant from 10 up to 300 nM. Similarly, BDNF (50 ng/ml) was able to reverse the MPP+ injuries. It could be mentioned that for the 3 highest test concentrations (30, 100 and 300 nM), bexarotene displayed a similar effect (in survival) as the one observed with BDNF used here as reference test compound. Similarly, a significant effect was observed on the length of TH neurite at the 2 highest concentrations of bexarotene (100 and 300 nM). The neurotrophic effect observed at the dose of 300 nM was as high as the one of BDNF (internal reference test compound). The results are illustrated in FIG. 15A, FIGS. 15B, and 15C. Representative images showing the regenerative effect of bexarotene on neurons are shown in FIG. 16.

### Example 9

### Bexarotene Doses that Cause Side Effects

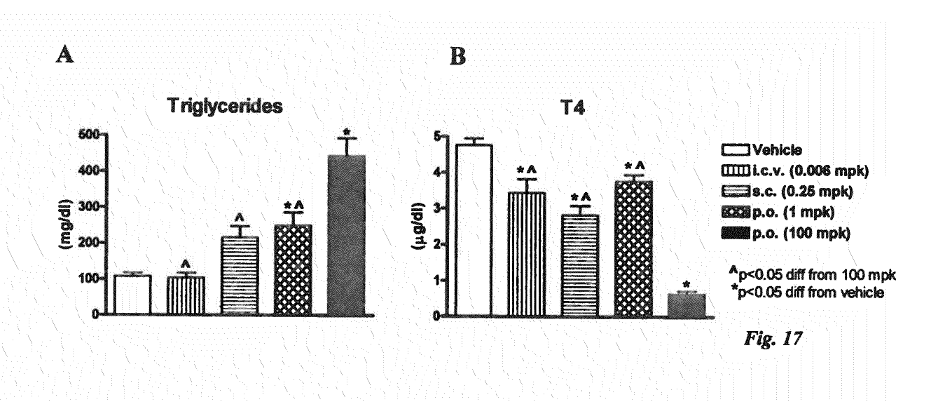

Elevation of serum triglycerides and hypothyroidism are two prominent side-effects known to be caused by bexarotene. Rats were administered bexarotene over a period of up to 8 days (i.c.v. or s.c.) or 5 days (oral), at doses previously shown to be effective in rat PD or cancer models (see Table 4), either with continuous infusion through the i.c.v. route (0.006 mg/kg/day), s.c. route (0.25 mg/kg/day) or orally (1 and 100 mg/kg/day). As shown in FIG. 17A, the triglyceride levels in rats given bexarotene i.c.v. or s.c. were not significantly different to vehicle treated animals. The triglyceride levels in rats given 1 mg/kg/day P.O. were significantly increased compared to vehicle. The triglyceride levels in all treatments (i.c.v., s.c. and 1 mg/kg/day p.o.) were significantly lower than in rats receiving 100 mg/kg/day p.o. Similarly, T4 levels were significantly higher in the i.c.v., s.c. and 1 mg/kg/day p.o. groups compared to the 100 mg/kg/day p.o. dose group (FIG. 17B). Finally, at higher doses of bexarotene a decrease in body weight gain was noted (see Table 5).

TABLE 5
Bexarotene was administered once per day orally, at the indicated
doses (mg/kg/day). Body weight was measured at the start and end
of the dosing period and expressed as percent body weight gain.

| PO dose | % BW gain |
|---|---|
| Bex (1) | 20.8 +/− 2.8 |
| Bex (3) | 19.8 +/− 3.4 |
| Bex (10) | 20.4 +/− 1.6 |
| Bex (30) | 11.2 +/− 0.8 |
| Bex (100) | 7.0 +/− 3.6 |
| Veh | 20.9 +/− 2.6 |

These data suggest it is possible to identify doses of bexarotene that are effective for reversing neurodegeneration that have greatly reduced side effects compared to how bexarotene is currently used clinically.

### Example 10

### Bexarotene Doses in Humans

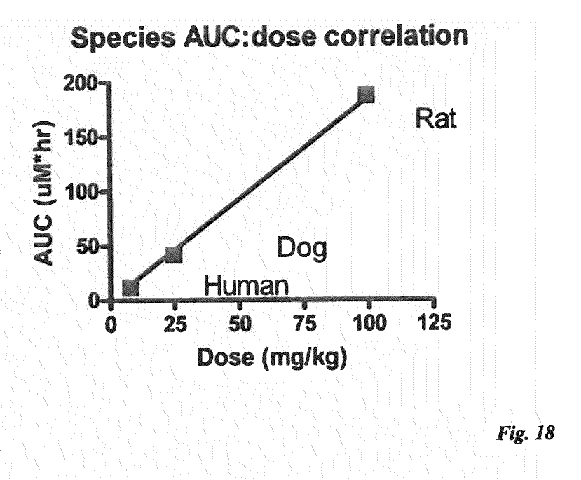

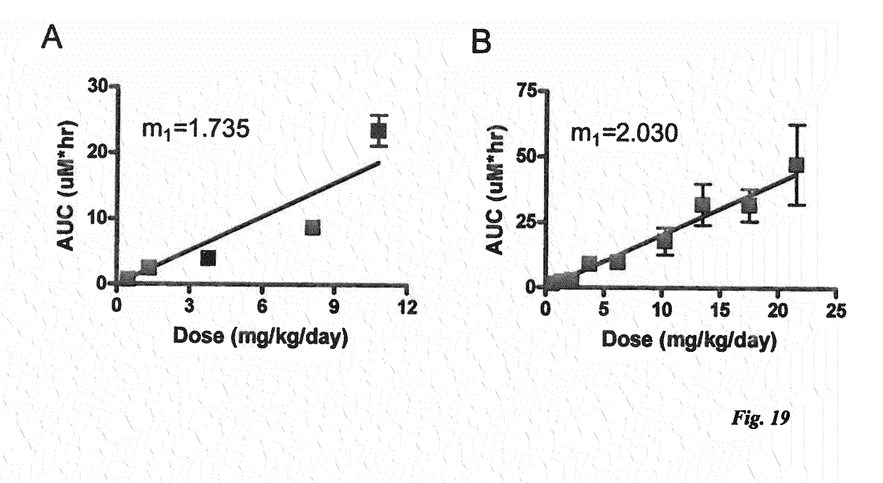

Extrapolation of AUC and dose between species. Information provided in Targretin NDA #21055 about the pharmacokinetics of bexarotene indicates that it is possible to extrapolate drug exposure (quantified as ‘area under the curve’ or AUC) between species (FIG. 18). Furthermore, published clinical data show that there is a strong correlation between doses of bexarotene given to humans, and AUC (FIG. 19).

Doses of bexarotene to effectively treat cancer in humans or rats. The recommended starting clinical dose of bexarotene for cancer treatment in humans is 300 mg/m²/day (equivalent to 8.1 mg/kg/day or ˜650 mg/day for an 80 kg person), and this dose may be increased if there is insufficient response (Targretin NDA #21055; Duvic et al., J. Clin. Oncol., 2001). The fully effective anti-cancer dose in rats is 100 mg/kg/day (Targretin NDA #21055).

Effective doses of bexarotene in a rat model of Parkinson's disease are much lower. We have shown bexarotene administered with continuous infusion through the intracerebroventricular route (C.I. i.c.v.) has regenerates neurons in rats previously given the neurotoxin 6OHDA (see FIG. 6-8). The brain concentration at this dose of bexarotene was 12 ng/g (see Table 4 above). Delivery of 0.25 mg/kg/day of bexarotene systemically using continuous infusion through the sub-cutaneous route (C.I. s.c.) provides a very similar bexarotene brain concentration of 14 ng/g (Table 4) and also effectively reversed behavioral deficits and regenerated neurons damaged by 6OHDA lesion (FIGS. 9, 10 and 11). Finally, once daily oral administration of 1 mg/kg/day of bexarotene also effectively reversed behavioral deficits and regenerated neurons damaged by 6OHDA lesion (FIGS. 12 and 13). Oral administration of 1 mg/kg/day of bexarotene provides brain concentrations greater than the threshold brain concentration of bexarotene needed for efficacy determined in the i.c.v. and s.c. experiments (FIG. 5). Thus, one can use the plasma levels of bexarotene delivered at 0.25 mg/kg/day C.I. s.c., which were 12 ng/ml, to calculate AUC in rats for an effective PD dose delivered systemically. Similarly, one can calculate the AUC of bexarotene administered at 1 mg/kg/day orally to determine the AUC in rats for an effective PD dose delivered orally.

Thus using the ratio of AUC in rats for an effective cancer dose to an effective PD dose, one may extrapolate the AUC observed in humans at effective cancer doses to the AUC needed for efficacy in PD. One can then estimate doses of bexarotene needed for efficacy against PD in humans using the human AUC/dose correlation(s) below.

Human data:

| | |
|---|---|
| Recommended anti-cancer dose: | 300 mg/m² = 8.1 mg/kg or 648 |
| | mg/day¹ |
| ²AUC at recommended | 11.6 μM*hr |
| anti-cancer dose: | |

Rat data:

| | |
|---|---|
| ³Effective anti-cancer dose: | 100 mg/kg/day (rats); |
| | 60 mg/kg/day (mice) |
| ³AUC at 30 mg/kg/day: | 24 μM*hr |
| AUC at 60 mg/kg/day (interpolated): | 33 μM*hr |
| ³AUC at 100 mg/kg/day: | 42 μM*hr |
| Effective PD dose: | 0.25 mg/kg/day (s.c.), |
| | 1 mg/kg/day (p.o.) |
| AUC at effective PD dose⁴: | 0.8 μM*hr (s.c.), |
| | 2.7 μM*hr (p.o.) |

¹based on an 80 kg person;
²AUC from Miller et al., 1997; Targretin NDA #21055, and Duvic et al., 2001 at the recommended anti-cancer dose of 300 mg/m²;
³from Targretin NDA #21055.
⁴AUC calculated using the trapezoidal method for s.c. dosing and using prizm software for p.o. dosing.
⁴AUC for p.o. represents the average of the AUCs determined for 6OHDA lesioned and intact rats (see Table 4).

Effective doses of bexarotene to treat Parkinson's disease in humans may be estimated as follows:

(Rat AUC_{Parkinson}÷Rat AUC_{cancer})×Human AUC_{cancer}=Human AUC_{Parkinson's}

Using the correlation of human AUC to human dose in FIG. 19A or 19B:

Human AUC_{Parkinson's}÷slope=Human Dose_{Parkinson's}

A summary of these calculations is provided in Table 6 below.

TABLE 6

| | | | Parkinson's | | Cancer | | Parkinson's | | |
|---|---|---|---|---|---|---|---|---|---|
| | Cancer (rat) | | (rat) | | (human) | | (human) | | |
| | Treatment: | | | | | | | | |
| | dose | AUC | dose | AUC | AUC | dose | AUC | dose | |
| | Units: | | | | | | | | |
| | mg/kg | (μM * hr) | mg/kg | (μM * hr) | (μM * hr) | mg/day | (μM * hr) | mg/day | |
| | Column: | | | | | | | | |
| | A | B | C | D | E | F | G | H | I |
| ORAL | 30 | 24 | 1 | 2.7 | 11.6 | 648 | 1.27 | 59 | 50 |
| | 60 | 33 | 1 | 2.7 | 11.6 | 648 | 0.93 | 43 | 37 |
| | 100 | 42 | 1 | 2.7 | 11.6 | 648 | 0.73 | 34 | 29 |
| s.c. infusion | 30 | 24 | 0.25 | 0.8 | 11.6 | 648 | 0.38 | 18 | 15 |
| | 60 | 33 | 0.25 | 0.8 | 11.6 | 648 | 0.28 | 13 | 11 |
| | 100 | 42 | 0.25 | 0.8 | 11.6 | 648 | 0.22 | 10 | 9 |

A: Rat effective dose (cancer) of 100 mg/kg = effective anti-cancer dose in rats (from Targretin NDA #21055).
B: Rat AUC (cancer) at 30 and 100 mg/kg P.O. from Targretin NDA #21055 and confirmed experimentally. Rat AUC (cancer) at 60 mg/kg interpolated from AUCs at 30 and 100 mg/kg.
C: Rat effective dose (PD) of 0.25 mg/kg administered as s.c. continuous infusion or 1 mg/kg/day QD p.o.
D: Rat plasma AUC (PD) calculated using the trapezoidal rule (s.c.) or prizm software (p.o.). AUC oral represents the average of the AUCs determined for 6OHDA lesioned and intact rats (see Table 4).
E: Human AUC (cancer) at 300 mg/m² P.O. (equivalent to 8.1 mg/kg) from values reported previously (Miller et al., 1997; Targretin NDA #21055, and Duvic et al., 2001).
F: Human starting dose (cancer) based on 300 mg/m² dose (8.1 mg/kg/day × 80 kg person = 648 mg/day).
G: Human AUC (PD) calculated as (human AUC_{cancer} × Rat AUC_{PD} ÷ Rat AUC_{cancer})
H: Human dose (PD) estimated using human AUC (PD) divided by m₁ (slope of FIG. 19A) × 80 kg
I: Human dose (PD) estimated using human AUC (PD) divided by m₂ (slope of FIG. 19B) × 80 kg

A second way to estimate human doses to treat PD is to compare extrapolated AUC values from Table 6 to actual AUC values measured in humans receiving low doses of bexarotene (Table 7).

TABLE 7
Human exposure to low dose Targretin (bexarotene).

| Dose | | | Measured AUC | Extrapolated AUC |
|---|---|---|---|---|
| (mg/m2) | (mg/kg) | *(mg) | (μM*hr) | (μM*hr) |
| ^{a}18 | 0.5 | 39 | 0.7-0.9 | 0.2-0.4 (s.c.) |
| ^{b}21 | 0.6 | 45 | 1.4 | and |
| ^{c}37 | 0.9 | 75 | 1.0-1.1 | 0.7-1.3 (p.o.) |

*based on an 80 kg person.
^{a}from Miller et al, 1997.
^{b}from Rizvi et al, 1999.
^{c}from Targretin summary basis of approval - see EMEA approval.
Extrapolated AUC are from Table 6.
s.c. is subcutaneous, p.o. is oral.

Examination of Table 7 reveals that the extrapolated AUC values are comparable to, or lower than the AUC values measured in humans receiving bexarotene doses of 39 to 75 mg or 0.5 to 0.9 mg/kg based on an 80 kg, 180 cm individual.

A third means to estimate doses to treat PD in humans is to use FDA recommended methods of extrapolating between human and animal dosing data. This method has also been described in the scientific literature (Reagan-Shaw et al., 2008). In this publication, methods are presented to calculate body surface area (BSA). The Km factor, body weight (kg) divided by BSA (m²), is calculated for rats and humans. The human equivalent dose in mg/kg is then calculated as rat dose×Kmᵣₐₜ÷Kmₕᵤₘₐₙ. A summary of these calculations is provided in Table 8.

TABLE 8
Human equivalent dose using FDA scaling guidelines

| | | | Human | | |
|---|---|---|---|---|---|
| | | | equivalent | | |
| | | Effective | dose | Human total dose | |
| | | dose | *(rat dose × Kmᵣₐₜ ÷ | s.c. or | |
| Therapeutic | Dosing | rat | Kmₕᵤₘₐₙ) | i.c.v. | oral |
| indication | route | (mg/kg/day) | (mg/kg/day) | (mg/day) | (mg/day) |
| Cancer | p.o. | 60 | 9.0 | — | 720 |
| PD i.c.v. | i.c.v. | 0.00625 | 0.001 | 0.08 | — |
| PD s.c. | s.c. | 0.25 | 0.038 | 3.0 | — |
| PD p.o. | p.o. | 1 | 0.150 | — | 12 |

*Kmᵣₐₜ and Kmₕᵤₘₐₙ values of 6 and 40, respectively, calculated as described (Reagan-Shaw et al., 2008). Human Km and Human total dose based on an 80 kg, 180 cm individual.

The method outlined in the FDA publication referenced above extrapolates an effective dose to treat cancer in rats of 60 mg/kg/day administered orally (see Targretin NDA #21055) to 720 mg/day in humans (see Table 8), which is in good agreement with the actual suggested starting dose of 648 mg/day (Targretin NDA #21055). The same method extrapolates the effective doses in the rat 6OHDA lesion model, which are 0.00625, 0.25 and 1 mg/kg/day administered i.c.v., s.c. and p.o., respectively, to 0.08, 3, and 12 mg/day to treat PD in humans.

### Intracerebroventricular (i.c.v.) Administration of Bexarotene Offers Additional Advantages

At least two additional benefits may be realized with i.c.v. administration of Bexarotene:
Very low doses will be effective
Brain:plasma ratio of drug will increase further reducing systemic drug exposure, and thus systemic side effects.

Table 4 (shown above) reveal that the brain/plasma ratio is higher with i.c.v. administration, reaching effective brain concentrations while keeping peripheral levels low.

The brain concentration achieved with 0.25 mg/kg/day of s.c. administration (12 ng/g); was also achieved with 0.00625 mg/kg/day of i.c.v. administration, a 40-fold lower dose. Significantly, this level of brain exposure had neuroregenerative effects in a rat model of PD (see FIG. 6-8, 14). Using a plasma level of 2 ng/ml (Table 4), the AUC with i.c.v. administration was at least 6-fold lower than with s.c. administration while providing equal brain exposure. Using the ranges provided for s.c. administration in Table 6, compared to the recommended dose for Targretin in humans to treat cancer (300 mg/m², equivalent to ˜650 mg/day for an 80 kg person) the effective dose of bexarotene administered i.c.v. to humans are estimated to be:
AUC basis (6×): effective i.c.v. dose of bexarotene is 1.5 to 3.0 mg/day
Dose basis (40×): effective i.c.v. dose of bexarotene is 0.25 to 0.5 mg/day

Several methods are presented above to extrapolate effective doses of bexarotene in a rat model of PD to doses to treat PD in humans. Based on these methods, the predicted dose ranges are:

| | |
|---|---|
| Oral administration | 12-59 mg/day |
| Subcutaneous infusion | 3-18 mg/day |
| Intracerebroventricular infusion | 0.08-3 mg/day |

Based on an 80 kg individual, the predicted dose ranges are:

| | |
|---|---|
| Oral administration | 0.15-0.74 mg/kg/day |
| Subcutaneous infusion | 0.04-0.23 mg/kg/day |
| Intracerebroventricular infusion | 0.001-0.04 mg/kg/day |

The dose ranges above thus span from about 0.08 mg/day to about 59 mg/day. Since these doses are predicted, the skilled person realises that somewhat lower and somewhat higher doses also will have desired effect. With some minor generalization, estimates based on the above predictions are as follows:

| | |
|---|---|
| Oral administration | 10-70 mg/day or 10-60 mg/day; or 0.13-0.88 |
| | mg/kg/day or 0.13-0.75 mg/kg/day |
| Subcutaneous infusion | 1-20 mg/day or 0.01-0.25 mg/kg/day |
| Intracerebroventricular | 0.05-5 mg/day or 0.0006-0.06 mg/kg/day |
| infusion | |

Thus, the above predicted doses clearly support the dose range from about 0.05 mg/day to about 75 mg/day. It is also clear that the doses will vary depending on the administration route used.

The invention should not be construed as limited to the dose ranges given in the examples. For example, the dose ranges based on mg/day may be increased or decreased to account for individual differences in body mass, which is well known to the skilled person and which is routine work for a physician; however the doses shall always be low to minimize undesired side effects. Dose ranges may also be affected by other factors such a patient compliance and individual patient response. Thus also dose ranges as used throughout the application are considered likely.

Although the invention has been described with reference to embodiments and examples, it should be understood that numerous and various modifications can be made without departing from the spirit of the invention. Accordingly, the invention is limited only by the following claims.

### REFERENCES

Carpentier, R., P. Sacchetti, et al. (2008). “The glucocorticoid receptor is a coregulator of the orphan nuclear receptor Nurr1.” J Neurochem 104(3): 777-89.
Castro, D. S., Arvidsson, M., Bondesson, B. M. and Perlmann, T. (1999) Activity of the Nurr1 carboxyl-terminal domain depends on cell type and integrity of the activation function 2. J. Biol. Chem., 274, 37483-37490.
Dubois, C., B. Hengerer, et al. (2006). “Identification of a potent agonist of the orphan nuclear receptor Nurr1.” Chem Med Chem 1(9): 955-8.
Duvic M, Hymes K, Heald P, Breneman D, Martin A G, Myskowski P, Crowley C, Yocum R C; Bexarotene Worldwide Study Group. Bexarotene is effective and safe for treatment of refractory advanced-stage cutaneous T-cell lymphoma: multinational phase II-III trial results. J Clin Oncol. 2001 May 1; 19(9):2456-71.
FDA recommended methods to extrapolate between human and animal dosing data: www.fda.gov/downloads/Drugs/GuidanceComplianceRegulatoryInformation/Guidances/ucm078932.pdf
Gniadecki R, Assaf C, Bagot M, Dummer R, Duvic M, Knobler R, Ranki A, Schwandt P, Whittaker S. The optimal use of bexarotene in cutaneous T-cell lymphoma. Br J Dermatol. 2007 September; 157(3):433-40.
Hou, J. G., Lin, L. F., & Mytilineou, C. (1996). Glial cell line-derived neurotrophic factor exerts neurotrophic effects on dopaminergic neurons in vitro and promotes their survival and regrowth after damage by 1-methyl-4-phenylpyridinium. J Neurochem., 66, 74-82.
Hung, H. C. & Lee, E. H. (1996). The mesolimbic dopaminergic pathway is more resistant than the nigrostriatal dopaminergic pathway to MPTP and MPP+ toxicity: role of BDNF gene expression. Brain Res Mol Brain Res., 41, 14-26.
Jankovic, J., S. Chen, et al. (2005). “The role of Nurr1 in the development of dopaminergic neurons and Parkinson's disease.” Prog Neurobiol 77(1-2): 128-38.
Lund B W, Piu F, Gauthier N K, Eeg A, Currier E, Sherbukhin V, Brann M R, Hacksell U, Olsson R. Discovery of a potent, orally available, and iso form-selective retinoic acid beta2 receptor agonist. J Med Chem. 2005 Dec. 1; 48(24):7517-9.
Hermanson, E., B. Joseph, et al. (2003). “Nurr1 regulates dopamine synthesis and storage in MN9D dopamine cells.” Exp Cell Res 288(2): 324-34.
Miller V A, Benedetti F M, Rigas J R, Verret A L, Pfister D G, Straus D, Kris M G, Crisp M, Heyman R, Loewen G R, Truglia J A, Warrell R P Jr. (1997) Initial clinical trial of a selective retinoid X receptor ligand, LGD1069. J Clin Oncol. 15:790-795.
Ohta K, Kawachi E, Inoue N, Fukasawa H, Hashimoto Y, Itai A, Kagechika H. Retinoidal pyrimidinecarboxylic acids. Unexpected diaza-substituent effects in retinobenzoic acids. Chem Pharm Bull (Tokyo). 2000 October; 48(10):1504-13.
Ordentlich, P., Y. Yan, et al. (2003). “Identification of the antineoplastic agent 6-mercaptopurine as an activator of the orphan nuclear hormone receptor Nurr1.” J Biol Chem 278(27): 24791-9.
Paisan-Ruiz, C., Jain, S., Evans, E. W., Gilks, W. P., Simon, J., van der, B. M., Lopez, d. M., Aparicio, S., Gil, A. M., Khan, N., Johnson, J., Martinez, J. R., Nicholl, D., Carrera, I. M., Pena, A. S., de Silva, R., Lees, A., Marti-Masso, J. F., Perez-Tur, J., Wood, N. W., & Singleton, A. B. (2004). Cloning of the gene containing mutations that cause PARK8-linked Parkinson's disease. Neuron., 44, 595-600.
Reagan-Shaw S, Nihal M, Ahmad N. (2008) Dose translation from animal to human studies revisited. FASEB J. 22:659-61.
Rizvi, N. A., Marshall, J. L. Dahut, W, Ness, E., Truglia, J. A., Loewen G., Gill, G. M., Ulm, E. H., Geiser, R., Jaunakis, D. & Hawkins, M. J. (1999). A Phase I Study of LGD1069 in Adults with Advanced Cancer. Clin. Cancer Res. 5:1658-1164
Sacchetti, P., Dwornik, H., Formstecher, P., Rachez, C. and Lefebvre, P. (2002) Requirements for heterodimerization between the orphan nuclear receptor nurr1 and retinoid X receptors. J. Biol. Chem., 277, 35088-35096.
Saijo, K., B. Winner, et al. (2009). “A Nurr1/CoREST pathway in microglia and astrocytes protects dopaminergic neurons from inflammation-induced death.” Cell 137(1): 47-59.
Schinelli, S., Zuddas, A., Kopin, I. J., Barker, J. L., & DI Porzio, U. (1988). 1-Methyl-4-phenyl-1,2,3,6-tetrahydropyridine metabolism and 1-methyl-4-phenylpyridinium uptake in dissociated cell cultures from the embryonic mesencephalon. J Neurochem., 50, 1900-1907.
Shi, Y. (2007). “Orphan nuclear receptors in drug discovery.” Drug Discov Today 12(11-12): 440-5.
Tan, P. K., J. Wang, et al. (2007). “Monitoring interactions between receptor tyrosine kinases and their downstream effector proteins in living cells using bioluminescence resonance energy transfer.” Mol Pharmacol 72(6): 1440-6.
Targretin NDA #21055. Information may be found at http://www.accessdata.fda.gov/drugsatfda_docs/nda/99/21055_Targretin.cfm. See Pharmacology Review, Part 1.
Targretin Summary Basis of Approval. Possible to obtain at the homepage of the European Medicines Agency (EMEA) by searching for Targretin. The document is referred to as Targretin: EPAR—Scientific Discussion and first published Aug. 8, 2006 by the EMEA.
Uhl, G. R., Javitch, J. A., & Snyder, S. H. (1985). Normal MPTP binding in parkinsonian substantial nigra: evidence for extraneuronal toxin conversion in human brain. Lancet., 1, 956-957.
Umemiya H, Fukasawa H, Ebisawa M, Eyrolles L, Kawachi E, Eisenmann G, Gronemeyer H, Hashimoto Y, Shudo K, Kagechika H. Regulation of retinoidal actions by diazepinylbenzoic acids. Retinoid synergists which activate the RXR-RAR heterodimers. J Med Chem. 1997 Dec. 19; 40(26):4222-34.
Vila, M. & Przedborski, S. (2004). Genetic clues to the pathogenesis of Parkinson's disease. Nat. Med., 10 Suppl, S58-S62.
Wagner C E, Jurutka P W, Marshall P A, Groy T L, van der Vaart A, Ziller J W, Furmick J K, Graeber M E, Matro E, Miguel B V, Tran I T, Kwon J, Tedeschi J N, Moosavi S, Danishyar A, Philp J S, Khamees R O, Jackson J N, Grupe D K, Badshah S L, Hart J W. Modeling, synthesis and biological evaluation of potential retinoid X receptor (RXR) selective agonists: novel analogues of 4-[1-(3,5,5,8,8-pentamethyl-5,6,7,8-tetrahydro-2-naphthyl)ethynyl]benzoic acid (bexarotene). J Med Chem. 2009 Oct. 8; 52(19):5950-66.
Wallen-Mackenzie, A., A. Mata de Urquiza, et al. (2003). “Nurr1-RXR heterodimers mediate RXR ligand-induced signaling in neuronal cells.” Genes Dev 17(24): 3036-47.
Zetterstrom, R. H., L. Solomin, et al. (1997). “Dopamine neuronagenesis in Nurr1-deficient mice.” Science 276(5310): 248-50.

1-57. (canceled)

58. A method for the treatment of a neurodegenerative disease or disorder, comprising the administration to a patient having a neurodegenerative disease or disorder an effective amount of the compound of formula (I) 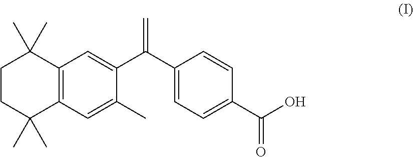 [SMILES: C=C(c1ccc(C(=O)O)cc1)c1cc2c(cc1C)C(C)(C)CCC2(C)C]
or a pharmaceutically acceptable salt, solvate, polymorph or hydrate thereof, wherein the compound is administered to the patient at a low dose.

59. The method of claim 58, wherein the compound is administered to the patient at a dose of about 0.05 mg to about 75 mg per day.

60. The method of claim 58, wherein the compound is administered to the patient at a dose of about 0.0006 mg to about 1 mg per kg per day.

61. The method of claim 58, wherein the compound is administered to the patient at a dose of about 0.05 mg to about 65 mg per day.

62. The method of claim 58, wherein the compound is administered to the patient at a dose of about 0.0006 mg to about 0.8 mg per kg per day.

63. The method of claim 58, wherein the compound is administered to the patient at a dose of about 0.05 mg to about 50 mg per day.

64. The method of claim 58, wherein the compound is administered to the patient at a dose of about 0.0006 mg to about 0.6 mg per kg per day.

65. The method of claim 58, wherein the compound is administered orally.

66. The method of claim 65, wherein the compound is administered in a dose of from about 10 to about 70 mg per day, such as from about 10 to about 60 mg per day, or such as from about 12 to about 59 mg per day.

67. The method of claim 65, wherein the compound is administered in a dose of from about 0.13 to about 0.88 mg per kg per day, such as from about 0.13 to about 0.75 mg per kg per day, or such as from about 0.15 to about 0.74 mg per kg per day.

68. The method of claim 58, wherein the compound is administered through a non-oral route of administration.

69. The method of claim 68, wherein the compound is administered subcutaneously.

70. The method of claim 69, wherein the compound is administered in a dose of from about 1 to about 20 mg per day, such as from about 3 to about 18 mg per day.

71. The method of claim 69, wherein the compound is administered in a dose of from about 0.01 to about 0.25 mg per kg per day, such as from about 0.04 to about 0.23 mg per kg per day.

72. The method of claim 68, wherein the compound is administered transdermally.

73. The method of claim 68, wherein the compound is administered intracerebroventricullarly.

74. The method of claim 73, wherein the compound is administered to the patient at a dose of about 0.05 mg to about 20 mg per day.

75. The method of claim 73, wherein the compound is administered to the patient at a dose of about 0.0006 mg to about 0.3 mg per kg per day.

76. The method of claim 73, wherein the compound is administered to the patient at a dose of about 0.05 mg to about 15 mg per day.

77. The method of claim 73, wherein the compound is administered to the patient at a dose of about 0.0006 mg to about 0.2 mg per kg per day.

78. The method of claim 73, wherein the compound is administered to the patient at a dose of from about 0.05 to about 5 mg per day, such as from about 0.08 to about 3 mg per kg per day.

79. The method of claim 73, wherein the compound is administered to the patient at a dose of from about 0.0006 to about 0.06 mg per kg per day, such as from about 0.001 to about 0.04 mg per kg per day.

80. The method of claim 68, wherein said compound is to be administered in a continuous infusion.

81. The method of claim 58, wherein the neurodegenerative disease is associated with a Nurr1 receptor.

82. The method of claim 58, wherein the neurodegenerative disease is Parkinson's disease.

83-111. (canceled)

112. A method of treating a neurodegenerative disease wherein a compound of formula (I) 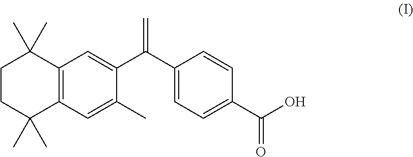 [SMILES: C=C(c1ccc(C(=O)O)cc1)c1cc2c(cc1C)C(C)(C)CCC2(C)C]
or a pharmaceutically acceptable salt, solvate, polymorph or hydrate thereof, is administered to a subject wherein the side effects associated with continuous treatment are low due to the administration of a low dose of compound of formula (I).

113-121. (canceled)
